SEQUENCE LISTING (17 sequences)

SEQ ID NO: 1 - DNA, 20 bp, Artificial Sequence
  Description of Artificial Sequence Synthetic primer
tggacagcacccgaagccat

SEQ ID NO: 2 - DNA, 23 bp, Artificial Sequence
  Description of Artificial Sequence Synthetic primer
gatgaccaaccagtgtgatccct

SEQ ID NO: 3 - DNA, 23 bp, Artificial Sequence
  Description of Artificial Sequence Synthetic primer
aaaaagctaaacgtggagcagcc

SEQ ID NO: 4 - DNA, 22 bp, Artificial Sequence
  Description of Artificial Sequence Synthetic primer
ccattgggtggagaggaaatcc

SEQ ID NO: 5 - DNA, 23 bp, Artificial Sequence
  Description of Artificial Sequence Synthetic primer
gattcctttttcctcctgaaccc

SEQ ID NO: 6 - DNA, 22 bp, Artificial Sequence
  Description of Artificial Sequence Synthetic primer
ttccagtagacagcgtagcggt

SEQ ID NO: 7 - DNA, 23 bp, Artificial Sequence
  Description of Artificial Sequence Synthetic primer
gattcctttttcctcctgaaccc

SEQ ID NO: 8 - DNA, 23 bp, Artificial Sequence
  Description of Artificial Sequence Synthetic primer
ccatgtagaggacatagcgctca

SEQ ID NO: 9 - DNA, 20 bp, Artificial Sequence
  Description of Artificial Sequence Synthetic primer
cgctgctgctcctgctgtta

SEQ ID NO: 10 - DNA, 23 bp, Artificial Sequence
  Description of Artificial Sequence Synthetic primer
ggaacttctccgagaacttgagc

SEQ ID NO: 11 - DNA, 20 bp, Artificial Sequence
  Description of Artificial Sequence Synthetic primer
cgctgctgctcctgctgtta

SEQ ID NO: 12 - DNA, 22 bp, Artificial Sequence
  Description of Artificial Sequence Synthetic primer
ctcgtacagggtctcgttggtc

SEQ ID NO: 13 - DNA, 31 bp, Artificial Sequence
  Description of Artificial Sequence Synthetic primer
cttgacagctaaatatctggataaagagatc

SEQ ID NO: 14 - DNA, 23 bp, Artificial Sequence
  Description of Artificial Sequence Synthetic primer
cattacacttccagacctgggac

SEQ ID NO: 15 - DNA, 20 bp, Artificial Sequence
  Description of Artificial Sequence Synthetic primer
ctaaggtcctattttgcctg

SEQ ID NO: 16 - DNA, 22 bp, Artificial Sequence
  Description of Artificial Sequence Synthetic primer
gtctgcagtcggagacttgcag

SEQ ID NO: 17 - DNA, 18 bp, Artificial Sequence
  Description of Artificial Sequence Synthetic primer
cttcgcagcctgcgcctc

(19) United States
(12) Patent Application Publication
(10) Pub. No.: US 2008/0031870 A1
(43) Pub. Date: Feb. 7, 2008

(54) Method of proliferation in neurogenic regions

(76) Inventors: Johan Holmberg, Stockholm (SE); Jonas Frisen, Stockholm (SE)

Correspondence Address: Ivor R. Elrifi;Mintz, Levin, Cohn, Ferris,, Glovsky and Popeo, P.C., One Financial Center, Boston, MA, 02111, US

(21) Appl. No.: 11/732,203

(22) Filed: Apr. 2, 2007

Related U.S. Application Data
(63) Continuation of application No. 10/291,290, filed on Nov. 8, 2002, now abandoned.
(60) Provisional application No. 60/345,206, filed on Nov. 9, 2001.
(60) Provisional application No. 60/393,272, filed on Jul. 2, 2002.

(51) Int. Cl. A61K 38/17; A61K 39/395; A61P 25/00

(52) U.S. Cl. 424/130.1; 514/12

(57) ABSTRACT

Novel methods for the use of modulators to modulate an activity of a neural stem cell or a neural progenitor cell in vivo or in vitro are provided. The disclosure provides novel methods for the treatment of neurological diseases and disorders.

This application claims the benefit of priority from U.S. 60/345,206 filed Nov. 9, 2001 and from U.S. 60/393,272 filed Jul. 2, 2002. Both applications are hereby incorporated by reference in their entirety.

### FIELD OF THE INVENTION

This application is directed to compounds that disrupt EphA7 and ephrin-A5 interaction or EphA7 and ephrin-A2 interaction. Further, this application is directed to methods for the use of these compounds and to the use of the compounds for the alleviation of one or more symptoms of a neurological disease or disorder.

### BACKGROUND OF THE INVENTION

Receptor tyrosine kinases (RTKs) are important mediators of effects from signalling proteins in both the developing and the adult organism. The Eph receptors constitute the largest family of RTKs. Ephrins are membrane-bound ligands for the Eph protein tyrosine kinase receptor family. This class of molecules is further subdivided into A-class and B-class ephrins that couple to A- and B-type receptors, respectively. One exception to this rule is EphA4, which elicits binding to both A and B ligands. Both classes of ligands are anchored to the membrane even though the A ligands only are attached to the outer leaflet of the membrane in contrast to the B ligands that span the entire membrane (Frisén, J. et al., 1999. EMBO J. 18: 5159-5165; Wilkinson, D. G., 2001. Nat Rev Neurosci 2(3): 155-64). It has been shown that in order to activate the receptor, the ligand has to be clustered into oligomers (Davis, S. et al., 1994. Science 266: 816-819). Upon binding to the ligand complex the receptor itself dimerizes, enabling cross-phosphorylation of the tyrosine kinase domains, thus triggering a signal transduction cascade. One feature of Eph-ephrin signalling is the bi-directional signalling made possible by the membrane-attached ligands. The bi-directional signalling allows the ligand to act as a receptor and vice versa. This type of reverse signalling is well established with regard to the ephrin-Bs Henkemeyer, M. et al., 1996. Nature 383: 722-725) and recent evidence suggests that the same is true for the ephrin-As (Davy, A. et al., 1999. Genes Dev. 13: 3125-3135; Huai, et al., 2001. J Biol Chem 276(9): 6689-94). Eph receptors and ephrins show widespread expression in the developing nervous system as well as in the adult central nervous system (CNS) (Frisen, J. et al., 1999. EMBO J. 18: 5159-5165). First shown to act as repellent guidance cues for growing axons, recent research has revealed an astounding functional versatility of ephrins and Eph receptors (Wilkinson, D. G., 2001. Nat Rev Neurosci 2(3): 155-64).

Sites of neurogenesis are retained in the adult brain. Among these, two locations exhibit high levels of Eph receptor and ephrin expression: the dentate gyrus of the hippocampus and the lateral ventricular wall. The exact identity of the stem cells residing in the SVZ remains to be proven. Evidence for an ependymal as well as a subependymal origin for the stem cells exists (Johansson, C. et al., 1999. Cell 96: 25-34; Doetsch, F. et al., 1999. Cell 97: 703-716). Nevertheless it is possible to dissect the lateral wall, dissociate the tissue and cultivate the stem cells as buoyant spheres, denominated neurospheres). The neurospheres have self-renewal capacity and the developmental potential to differentiate into neurons, oligodendrocytes and astrocytes (Johansson, C. et al., 1999. Cell 96: 25-34). In vivo the stem cells give rise to neural progenitors that migrate along the lateral wall and feed into the rostromigratory stream, eventually ending up in the olfactory bulb (Doetsch, F. et al., 1996. Science 271: 978-981). To keep the cells in a low proliferative, undifferentiated mode one could postulate a non-autonomous mechanism where an extracellular protein could, when activated through binding to a ligand/receptor, act as a repressor on proliferation and/or differentiation. The lack of such an activation would results in increased proliferation or differentiation. The high expression of ephrin-A2, and EphA7 in the above mentioned neurogenic regions could be an indication of such a model.

### BRIEF SUMMARY OF THE INVENTION

The Eph tyrosine kinase receptors and their ephrin ligands confer short range communication between cells in the developing organism regulating diverse processes such as axon guidance, cell migration and neural tube formation (Wilkinson, D. G., 2001. Nat Rev Neurosci 2(3): 155-64). Even though both receptors and ligands are widely expressed in the adult nervous system, the knowledge concerning their roles in the adult is limited. Neurogenic areas in the adult brain, including the lateral wall of the lateral ventricle and the dentate gyrus of the hippocampus, express EphA7 and the ligands ephrin-A2. Mice lacking the receptor EphA7 exhibit increased cellular proliferation in the tissue on the lateral side of the lateral ventricle. We show that in the wild type organism the ephrin or Eph are negative regulators of proliferation, keeping it at a basal level. This effect involves reversed signalling through the ligand upon binding to the EphA7 receptor. Upon injection of the freely soluble form of ephrin-A5-Fc, ephrin-A2 or EphA7 either as monomers or as oligomers into the lateral ventricle, the number of proliferating cells as measured by BrdU-labelling was significantly higher than in sham injected mice. The ephrin-A5-Fc, ephrin-A2 or EphA7 proteins presumably disrupt the binding between the endogenous ligands and receptors, thus blocking signalling through the ligands and allowing a higher rate of proliferation.

Mice lacking EphA7 have minimal and compressed lateral ventricles due to increased amount of tissue in the lateral side of the ventricle. In the EphA7 null mutants BrdU injections show that the rate of proliferation in the ventricular wall is significantly higher than in the wild type. We have also performed in vitro studies that show a dramatic decrease in proliferation and/or differention capacity of neurospheres that are grown on a surface coated with EphA7 proteins in a conformation that can activate the ephrin ligands (clustered) whereas the opposite is true when EphA7 is presented in a form that will only block the ligands and not activate them (unclustered). The latter case mimics the mouse mutants with the coated EphA7 blocking the endogenous binding of EphA7 to ephrin-A2 in the neurospheres thus silencing the repressing activity of the ephrin-A ligand. Furthermore, when cultivated, stem cells from the lateral ventricular wall of an EphA7 null mutant mouse give rise to significantly higher numbers of spheres than corresponding tissue from a wild type mouse. We delivered ephrin-A5 or ephrin-A2 ligands through intracranial infusion into rodent lateral ventricle and measured proliferation in the lateral wall through BrdU labeling of dividing cells. We reasoned that the endogenous binding between EphA7 and the ephrin ligands would be interrupted and allow a higher rate of proliferation. This turned out to be the case as the number of proliferative cells was significantly increased in comparison with sham-injected animals. The interpretation that we believe best fits our data is one in which the ephrin-A2 are activated upon binding the EphA7 receptors. The activated ligand suppresses proliferation in the stem cell population, whereas if this activation is blocked, the proliferation is increased. When expressed within the same cell population as the full-length EphA7 receptor, a truncated splice form lacking the intracellular tyrosine kinase could act as a dominant negative EphA7 receptor, silencing the repellent activity of the ligand-bound full-length EphA7 (Holmberg, J. et al., 2000. Nature 408: 203-206). Furthermore, after intracranial infusion of ephrin-A5, we observed more BrdU positive cells in the olfactory bulb indicating the presence of functional neurogenesis by the increasing the number of stem cells in the neurogenic regions.

One embodiment of the invention is directed to a method of alleviating a symptom of a disease or disorder of the nervous system. In the method, a modulator that can modulate an activity of a neural stem cell or a neural progenitor cell is administered in vivo to a patient suffering from the disease or disorder of the nervous system. The term “modulator” is defined as a compound that can disrupt an interaction between EphA7 and ephrin-A5 or an interaction between EphA7 and ephrin-A2.

All the methods of the invention may use the following dosage range for administration of the modulator. The modulator may be administered in the dosage range of 0.1 ng/kg/day to 10 mg/kg/day; preferably about 1 ng/kg/day to 10 mg/kg/day; more preferably about 1 ng/kg/day to 5 mg/kg/day; and in particular about 0.1 μg/kg/day to 5 mg/kg/day. In another method of dosage, the modulator may be administered so that a target tissue achieve a modulator concentration of 0.1 nM to 50 nM. The target tissue (for any of the methods of this invention that refer to target tissue for administration) may be selected from the group consisting of tissue adjacent to the lateral ventricular wall, hippocampus, alveus, striatum, substantia nigra, retina, nucleus basalis of Meynert, spinal cord and cortex. In particular, the targeted tissue may be a region of the brain damaged by a disorder, stroke, or ischemia. One method of accomplishing this is to administer the modulator to a patient, determine the concentration of the modulator in the target tissue, and then depending on the outcome of the concentration measurement, decide on whether to continue to administer the modulator. Further, as the concentration is decreased over time, additional administration and measurements may be made.

The neural stem cell or neural progenitor cell referred to in this application may be a cell that is isolated from adult bone marrow, spinal cord, epithelial skin, epithelial intestinal, pancreas, hemapoetic system, blood, umbilical cord and muscle. In this embodiment, neural stem cell or neural progenitor cell is not limited to cells only found in an adult nervous system. For example, a puripotent stem cell may be isolated from the tissues listed and contact with the modulator may cause, directly or indirectly, the stem cell to become a neural stem cell or neural progenitor cell. As a non limiting illustration of this concept, an embryonic stem cell is the ultimate puripotent stem cell and yet it is not found in adult neuro tissue. Further examples would include the reported isolation of puripotent stem cells of the immune system that have been found in body fat. Thus, a neural stem cell or neural progenitor cell that can be derived from a pluripotent stem cell contacted to the modulator is also considered to be a neural stem cell or neural progenitor cell of this patent. Naturally, neural stem cell or neural progenitor cell is derived from tissue enclosed by dura mater, peripheral nerves or ganglia are of particular interest and is contemplate in the definition of all references to “neural stem cell or neural progenitor cell” in this application.

All the methods of this disclosure that involve modulator administration may use the following methods. The modulators may be administered orally or by injection. The term injection, throughout this application, encompasses all forms of injection known in the art and at least the more commonly described injection methods such as subcutaneous, intraperitoneal, intramuscular, intracerebroventricular, intraparenchymal, intrathecal and intracranial injection.

The modulator may be, for example, a EphA7 protein or a soluble fragment or an extra-cellular fragment of EphA7. Similarly, the modulator may be ephrin-A2 or ephrin-A5 or a soluble fragment or an extra-cellular fragment of these two proteins.

Where administration is by means other than injection, all known means are contemplated including administration by through the buccal, nasal or rectal mucosa. Commonly known delivery systems include administration by peptide fusion to enhance uptake or by via micelle delivery system.

Any of the methods of the invention may be used to alleviate a symptom of a diseases such as neurodegenerative disorders, neural stem cell disorders, neural progenitor disorders, ischemic disorders, neurological traumas, affective disorders, neuropsychiatric disorders and learning and memory disorders. Disease or disorder of the nervous system may be Parkinson's disease and Parkinsonian disorders, Huntington's disease, Alzheimer's disease, amyotrophic lateral sclerosis, spinal ischemia, stroke (including ischemic stroke), spinal cord injury and brain/spinal cord injury (especially cancer related brain/spinal cord injury). Disease or disorder of the nervous system may be schizophrenia, psychoses, depression, bipolar depression/disorder, anxiety syndromes/disorders, phobias, stress and related syndromes, cognitive function disorders, aggression, drug and alcohol abuse, obsessive compulsive behaviour syndromes, seasonal mood disorder, borderline personality disorder, cerebral palsy, multi-infarct dementia, Lewy body dementia, age related/geriatric dementia, epilepsy and injury related to epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy, retinal degenerative disorders, injury or trauma to the retina and sleep disorders. The complete and permanent treatment of the above diseases are also contemplated.

The term “neural stem cell or neural progenitor cell activity” includes activities such as proliferation, differentiation, migration or survival.

Another embodiment of the invention is directed to a method of modulating ephrin receptor or an ephrin ligand on the surface of a neural stem cell or neural progenitor cell. In the method, such cells expressing the receptor, or ligand are contacted to exogenous reagent, antibody, or affibody, wherein the exposure induces the neural stem cell or neural progenitor cell to proliferation, differentiation, migration or survival. The antibody may be a monoclonal (including a mixture of different monoclonals) or a polyclonal antibody. As described above, the neural stem cell or neural progenitor cell may be derived from fetal brain, adult brain, neural cell culture or a neurosphere.

Another embodiment of the invention is directed to a method of determining an isolated candidate ephrin receptor modulator or an isolated candidate ephrin ligand modulator for its ability to modulate neural stem cell or neural progenitor cell activity. The steps of the method included (a) administering said isolated candidate compound to a non-human mammal and (b) determining if the candidate compound has an effect on modulating the neural stem cell or neural progenitor cell activity in the non-human, mammal. The neural stem cell or neural progenitor cell is a cell that can be isolated from adult bone marrow, spinal cord, epithelial skin, epithelial intestinal, pancreas, hemapoetic system, blood, umbilical cord and muscle. Further the neural stem cell or neural progenitor cell may be derived from a pluripotent stem cell contacted to said modulator (details concerning the neural cells are described in previous paragraphs). The determining step may be comparing the neurological effects of said non-human mammal with a referenced non-human mammal not administered the candidate compound. The compound may be any compound that has the described effect. For example, the compound may be a peptide, a small molecule, a soluble receptor a receptor agonist and a receptor antagonist. In a preferred embodiment, the compound is (1) EphA7; (2) ephrin-A2; (3) ephrin-A5; (4) a soluble fragment of (1) (2) or (3); or an extra-cellular fragment of (1), (2) or (3).

Another embodiment of the invention is directed to a method for reducing a symptom of a disease or disorder of the central nervous system in a mammal in need of such treatment. In the method, an ephrin receptor or ephrin ligand modulator (i.e., the “modulator” as defined previously) is administered to the mammal, wherein the modulator disrupts an interaction between EphA7 and ephrin-A5 or an interaction between EphA7 and ephrin-A2. It should be noted that while the patent refer to an ephrin receptor modulator or ephrin ligand modulator, it is also contemplated that in some cases a compound may be both a ephrin receptor modulator and a ephrin ligand modulator. The useful dosages, including dosage to achieve a tissue concentration, and physical methods (injection etc.) of dosage administration are as previously described for all methods involving modulator administration. The targeted tissue includes tissue adjacent to the lateral ventricular wall, hippocampus, alveus, striatum, substantia nigra, retina, nucleus basalis of Meynert, spinal cord and cortex, and a region of the brain damaged by a disorder, stroke, or ischemia (as described in detail in the beginning of this section). The modulator may be selected from the group consisting of an antibody, an affibody, a small molecule and a receptor. Any of the method previously described may also be used in this embodiment for administration. For example, administration may be local or systemic.

In addition, administration of the modulator, in any of the methods of this disclosure, may include the details described in this paragraph. The modulator administration may be accompanied by administration of a ventricle wall permeability enhancer that is delivered before, during or after administration of ephrin receptor modulator or ephrin ligand modulator. As necessary or desired, the modulator may be admixed with a pharmaceutically acceptable carrier. Other reagents that may be administered before, during or after modulator administration include stem cell mitogens, survival factors, glial-lineage preventing agents, anti-apoptotic agents, anti-stress medications, neuroprotectants, anti-pyrogenics and a combination thereof.

Another embodiment of the invention is directed to a method for inducing the in situ proliferation differentiation, survival or migration of a neural stem cell or neural progenitor cell located in the neural tissue of a mammal. The method comprises administering a therapeutically effective amount of a modulator to the neural tissue, wherein the modulator disrupts an interaction between EphA7 and ephrin-A5 or an interaction between EphA7 and ephrin-A2. The administration of the modulator may be systemic or local. The administration may be used to alleviates a symptom of a diseases or disorders of the nervous system which include any disease or disorder listed above for other methods of the invention.

Another embodiment of the invention is directed to a method for accelerating the growth of neural stem cells or neural progenitor cells in a desired target tissue in a subject, comprising administering intramuscularly to the subject an expression vector containing an ephrin gene in a therapeutically effective amount. The expression vector may be a non-viral expression vector encapsulated in a liposome.

Another embodiment of the invention is directed to a method of enhancing neurogenesis in a patient suffering from a disease or disorder of the central nervous system, by intraventricular infusion of a modulator which disrupts an interaction between EphA7 and ephrin-A5 or an interaction between EphA7 and ephrin-A2. The disease or disorder may be neurodegenerative disorders, neural stem cell disorders, neural progenitor disorders, ischemic disorders, neurological traumas, affective disorders, neuropsychiatric disorders and learning and memory disorders.

Another embodiment of the invention is directed to a method for producing a population of cells enriched for human neural stem cells or human neural progenitor cells which can initiate neurospheres. The method comprises the steps of (a) contacting a population containing neural stem cells or neural progenitor cells with a reagent that recognizes a determinant on ephrin receptor; and (b) selecting for cells in which there is contact between the reagent and the determinant on the surface of the cells of step (a), to produce a population highly enriched for central nervous system stem cells. The reagent may be a soluble receptor, a small molecule, a peptide, an antibody and an affibody. The antibody may be a monoclonal or a polyclonal antibody. The population containing neural stem cells or neural progenitor cells may be obtained from any population of cells which gives rise to neural tissue. The neurotissue may be from a fetal brain or an adult brain.

Another embodiment of the invention is directed to a method for treating a disease or disorder of the central nervous system. In the method, a population of cells as described in the previous paragraph is administered to a mammal in need of the treatment. This include mammals (such as humans) with the disease or disorder. Another embodiment of the invention is directed to a non-human mammal engrafted with the enriched human neural stem cells or neural progenitor cells as described in the previous paragraph.

Examples of nonhuman mammals referred to in this disclosure include rats, mice, rabbits, horses, sheep, pigs and guinea pigs. The disease or disorders described are not limited to nonhumans and would include humans. Thus, naturally, references to patients include humans and other non human animals.

Another embodiment of the invention is directed to a method of activating an ephrin receptor on a neural stem cell or neural progenitor cell, the method comprising exposing a neural stem cell or neural progenitor cell expressing a receptor to exogenous reagent, antibody, or affibody, wherein the exposure induces the neural stem cell or neural progenitor cell to proliferate or differentiate. The antibody may be a monoclonal or a polyclonal antibody. The neural stem cell or neural progenitor cell may be derived from fetal brain, adult brain, neural cell culture or a neurosphere.

Another embodiment of the invention is directed to a method of reducing a symptom of a disease or disorder of the central nervous system in a subject comprising the steps of administering into the spinal cord of the subject a composition comprising a population of isolated primary neurons obtained from a fetus; and an ephrin receptor modulator such that the symptom is reduced.

Another embodiment of the invention is directed to a method of gene delivery and expression in a target cell of a mammal. The steps of the method include introducing a viral vector into the target cell, wherein the viral vector has at least one insertion site containing a nucleic acid encoding for EphA7, ephrin-A5, ephrin-A2, a soluble fragment thereof, or an extra-cellular fragment thereof; the nucleic acid gene operably linked to a promoter capable of expression in the host. The viral vector may be a non-lytic viral vector.

Another embodiment of the invention is directed to a method of gene delivery and expression in a target cell of a mammal. The steps of the method include (a) providing an isolated nucleic acid fragment encoding EphA7, ephrin-A5, or ephrin-A2 a soluble fragment thereof, or an extra-cellular fragment thereof; (b) selecting a viral vector with at least one insertion site for insertion of the isolated nucleic acid fragment operably linked to a promoter capable of expression in the target cells; (c) inserting the isolated nucleic acid fragment into the insertion site, and (d) introducing the vector into the target cell wherein the gene is expressed at detectable levels. The virus may be a retrovirus, adenovirus, or pox virus. One preferred pox virus is vaccinia. Other viruses include retrovirus, adenovirus, iridoviruses, coronaviruses, togaviruses, caliciviruses picornaviruses, adeno-associated viruses and lentiviruses. All the viruses may be from a strain that has been genetically modified or selected to be non-virulent in a host.

Another embodiment of the invention is directed to a method for alleviating a symptom of a disease or disorder of the central nervous system in a patient. The method involves the steps of (a) providing a population of neural stem cells or neural progenitor cells; (b) suspending the neural stem cells or neural progenitor cells in a solution comprising a mixture comprising an ephrin receptor modulator to generate a cell suspension; and (c) delivering the cell suspension to an injection site in the central nervous system of the patient to alleviate the symptom. An optional addition step may include the step of injecting the injection site with the growth factor for a period of time before, after, or during (coinjection) the step of delivering the cell suspension.

### BRIEF DESCRIPTION OF DRAWINGS

FIG. 1 depicts mRNA expression and immuno staining of (a) Ephrin-A2-Fc staining of the elateral ventricular wall; (b) In situ hybridization showing mRNA for the EphA7-gene; (d) EphA7-Fc staining of the elateral ventricular wall; and (e) EphA7-Fc staining of the lateral ventricular wall.

FIG. 2 depicts RT-PCR results from cultured human stem cells.

FIG. 3 (h) depicts the strategy for the targeted disruption of the EphA7 gene; (i) genotype analysis of EphA7 homozygous (+/+) and heterozygous (+/−) ES cells before (upper left panel) and after (upper right panel) the transfection with the Cre recombinase expression plasmid. Genomic DNA was isolated, digested with EcoRI and subjected to Southern Blot analysis using 3′ external probe shown in A. Alleles bearing the ephA7 mutation show a 6.8 kb band whereas a 9.7 kb band is observed in the wild type alleles. For PCR analysis, primer pairs amplifying a 3.6 kb (lower left panel, see also A) or a 0.5 kb (lower right panel) band in the case of successful recombination were used; (j) RT-PCR analysis of total RNA isolated from brain of adult animals of the indicated genotypes. Primers were chosen to amplify part of exon I of EphA7 (314 bp), (−) denoted no template control; (k) ventricular tissue architecture of an EphA7−/− mouse; (m) ventricular tissue architecture of a wild type mouse. In all figures, lateral is to the left and dorsal is up.

FIG. 4 depicts in vitro proliferation of neurospheres.

FIG. 5 depicts that EphA7 knockout mice have increased cell proliferation.

FIG. 6 depicts the quantification of an increased in the number of BrdU positive cells (proliferation) in ephrin-A2-Fc infused animals.

FIG. 7 depicts Ephrin-A5-Fc treatment indicates an increased proliferation and neurogenesis in the olfactory bulb in comparison to negative control (vehicle treated animals).

FIG. 8 depicts that EphA7 knockout mice have increased number of cells in the cortex.

### DETAILED DESCRIPTION OF INVENTION

It has been discovered that certain reagents are capable of modulating the differentiation, migration, proliferation and survival of neural stem/progenitor cells both in vitro and in vivo. As used herein, the term “modulate” refers to having an affect in such a way as to alter the differentiation, migration, proliferation and survival of neural stem cell (NSC) or neural progenitor cell (NPC) activity. Since undifferentiated, pluripotent stem cells can proliferate in culture for a year or more, the invention described in this disclosure provides an almost limitless supply of neural precursors.

As used herein, the term “neural stem cells” (NSCs) can be identified by their ability to undergo continuous cellular proliferation, to regenerate exact copies of themselves (self-renew), to generate a large number of regional cellular progeny, and to elaborate new cells in response to injury or disease. The terms “neural progenitor cells” or “neural precursor cells” (NPCs) mean cells that can generate progeny that are either neuronal cells (such as neuronal precursors or mature neurons) or glial cells (such as glial precursors, mature astrocytes, or mature oligodendrocytes). Typically, the cells express some of the phenotypic markers that are characteristic of the neural lineage. Typically, they do not produce progeny of other embryonic germ layers when cultured by themselves in vitro unless dedifferentiated or reprogrammed in some fashion.

As used herein, the term “reagent” refers to any substance that is chemically and biologically capable of activating a receptor, including peptides, small molecules, antibodies (or fragments thereof), affibodies and any molecule that dimerizes or multimerizes the receptors or replaces the need for activation of the extracellular domains. In one embodiment, the reagent is a small molecule.

As used herein, the term “antibody” as used in this disclosure refers to both polyclonal and monoclonal antibody. The ambit of the term deliberately encompasses not only intact immunoglobulin molecules, but also such fragments and derivatives of immunoglobulin molecules (such as single chain Fv constructs, diabodies and fusion constructs) as may be prepared by techniques known in the art, and retaining a desired antibody binding specificity. The term “affibody” (U.S. Pat. No. 5,831,012) refers to highly specific affinity proteins that can be designed to bind to any desired target molecule. These antibody mimics can be manufactured to have the desired properties (specificity and affinity), while also being highly robust to withstand a broad range of analytical conditions, including pH and elevated temperature. The specific binding properties that can be engineered into each capture protein allow it to have very high specificity and the desired affinity for a corresponding target protein. A specific target protein will thus bind only to its corresponding capture protein. The small size (only 58 amino acids), high solubility, ease of further engineering into multifunctional constructs, excellent folding and absence of cysteines, as well as a stable scaffold that can be produced in large quantities using low cost bacterial expression systems, make affibodies superior capture molecules to antibodies or antibody fragments, such as Fab or single chain Fv (scFv) fragments, in a variety of Life Science applications.

Preferred reagents of the invention include EphA7, ephrin-A5 or ephrin-A2 and any molecule that can interfere with EphA7 and ephrin-A5 interaction or EphA7 and ephrin-A2 interaction. The invention provides a method for in vivo disruption of EphA7/ephrin-A5 interaction or EphA7/ephrin-A2 activity and for therapeutic administration of EphA7, ephrin-A5 or ephrin-A2 and drug screening. In a preferred embodiment, the neural tissue is fetal or adult brain. In yet another embodiment, the population containing neural or neural-derived cells is obtained from a neural cell culture or neurosphere.

Production of Reagents

Reagents for treatment of patients are recombinantly produced, purified and formulated according to well known methods.

Reagents of the invention, and individual moieties or analogs and derivatives thereof, can be chemically synthesized. A variety of protein synthesis methods are common in the art, including synthesis using a peptide synthesizer. See, e.g., Peptide Chemistry, A Practical Textbook, Bodasnsky, Ed. Springer-Verlag, 1988; Merrifield, Science 232: 241-247 (1986); Barany, et al, Intl. J. Peptide Protein Res. 30: 705-739 (1987); Kent, Ann. Rev. Biochem. 57:957-989 (1988), and Kaiser, et al, Science 243: 187-198 (1989). The peptides are purified so that they are substantially free of chemical precursors or other chemicals using standard peptide purification techniques. The language “substantially free of chemical precursors or other chemicals” includes preparations of peptide in which the peptide is separated from chemical precursors or other chemicals that are involved in the synthesis of the peptide. In one embodiment, the language “substantially free of chemical precursors or other chemicals” includes preparations of peptide having less than about 30% (by dry weight) of chemical precursors or non-peptide chemicals, more preferably less than about 20% chemical precursors or non-peptide chemicals, still more preferably less than about 10% chemical precursors or non-peptide chemicals, and most preferably less than about 5% chemical precursors or non-peptide chemicals.

Chemical synthesis of peptides facilitates the incorporation of modified or unnatural amino acids, including D-amino acids and other small organic molecules. Replacement of one or more L-amino acids in a peptide with the corresponding D-amino acid isoforms can be used to increase the resistance of peptides to enzymatic hydrolysis, and to enhance one or more properties of biologically active peptides, i.e., receptor binding, functional potency or duration of action. See, e.g., Doherty, et al., 1993. J. Med. Chem. 36: 2585-2594; Kirby, et al., 1993, J. Med. Chem. 36:3802-3808; Morita, et al., 1994, FEBS Lett. 353: 84-88; Wang, et al., 1993 Int. J. Pept. Protein Res. 42: 392-399; Fauchere and Thiunieau, 1992. Adv. Drug Res. 23: 127-159.

Introduction of covalent cross-links into a peptide sequence can conformationally and topographically constrain the peptide backbone. This strategy can be used to develop peptide analogs of reagents with increased potency, selectivity and stability. A number of other methods have been used successfully to introduce conformational constraints into peptide sequences in order to improve their potency, receptor selectivity and biological half-life. These include the use of (i) C_{α}-methylamino acids (see, e.g., Rose, et al., Adv. Protein Chem. 37: 1-109 (1985); Prasad and Balaram, CRC Crit. Rev. Biochem., 16: 307-348 (1984)); (ii) N_{α}-methylamino acids (see, e.g., Aubry, et al., Int. J. Pept. Protein Res., 18: 195-202 (1981); Manavalan and Momany, Biopolymers, 19: 1943-1973 (1980)); and (iii) α,β-unsaturated amino acids (see, e.g., Bach and Gierasch, Biopolymers, 25: 5175-S192 (1986); Singh, et al., Biopolymers, 26: 819-829 (1987)). These and many other amino acid analogs are commercially available, or can be easily prepared. Additionally, replacement of the C-terminal acid with an amide can be used to enhance the solubility and clearance of a peptide.

Alternatively, a reagent may be obtained by methods well-known in the art for recombinant peptide expression and purification. A DNA molecule encoding the protein reagent can be generated. The DNA sequence is known or can be deduced from the protein sequence based on known codon usage. See, e.g., Old and Primrose, Principles of Gene Manipulation 3^{rd} ed., Blackwell Scientific Publications, 1985; Wada et al., Nucleic Acids Res. 20: 2111-2118(1992). Preferably, the DNA molecule includes additional sequence, e.g., recognition sites for restriction enzymes which facilitate its cloning into a suitable cloning vector, such as a plasmid. Nucleic acids may be DNA, RNA, or a combination thereof. Nucleic acids encoding the reagent may be obtained by any method known within the art (e.g., by PCR amplification using synthetic primers hybridizable to the 3′- and 5′-termini of the sequence and/or by cloning from a cDNA or genomic library using an oligonucleotide sequence specific for the given gene sequence, or the like). Nucleic acids can also be generated by chemical synthesis.

Any of the methodologies known within the relevant art regarding the insertion of nucleic acid fragments into a vector may be used to construct expression vectors that contain a chimeric gene comprised of the appropriate transcriptional/translational control signals and reagent-coding sequences. Promoter/enhancer sequences within expression vectors may use plant, animal, insect, or fungus regulatory sequences, as provided in the invention.

A host cell can be any prokaryotic or eukaryotic cell. For example, the peptide can be expressed in bacterial cells such as E. coli, yeast, insect cells, fungi or mammalian cells (such as Chinese hamster ovary cells (CHO) or COS cells). Other suitable host cells are known to those skilled in the art. In one embodiment, a nucleic acid encoding a reagent is expressed in mammalian cells using a mammalian expression vector. Examples of mammalian expression vectors include pCDM8 (Seed (1987) Nature 329:840) and pMT2PC (Kaufman et al. (1987) EMBO J 6: 187-195).

The host cells, can be used to produce (i.e., overexpress) peptide in culture. Accordingly, the invention further provides methods for producing the peptide using the host cells of the invention. In one embodiment, the method comprises culturing the host cell of invention (into which a recombinant expression vector encoding the peptide has been introduced) in a suitable medium such that peptide is produced. The method further involves isolating peptide from the medium or the host cell. Ausubel et al., (Eds). In: Current Protocols in Molecular Biology. J. Wiley and Sons, New York, N.Y. 1998.

An “isolated” or “purified” recombinant peptide or biologically active portion thereof is substantially free of cellular material or other contaminating proteins from the cell or tissue source from which the peptide of interest is derived. The language “substantially free of cellular material” includes preparations in which the peptide is separated from cellular components of the cells from which it is isolated or recombinantly produced. In one embodiment, the language “substantially free of cellular material” includes preparations of peptide having less than about 30% (by dry weight) of peptide other than the desired peptide (also referred to herein as a “contaminating protein”), more preferably less than about 20% of contaminating protein, still more preferably less than about 10% of contaminating protein, and most preferably less than about 5% contaminating protein. When the peptide or biologically active portion thereof is recombinantly produced, it is also preferably substantially free of culture medium, i.e., culture medium represents less than about 20%, more preferably less than about 10%, and most preferably less than about 5% of the volume of the peptide preparation.

The invention also pertains to variants of a reagent that function as either agonists (mimetics) or as antagonists. Variants of a reagent can be generated by mutagenesis, e.g., discrete point mutations. An agonist of a reagent can retain substantially the same, or a subset of, the biological activities of the naturally occurring form of the reagent. An antagonist of the reagent can inhibit one or more of the activities of the naturally occurring form of the reagent by, for example, competitively binding to the receptor. Thus, specific biological effects can be elicited by treatment with a variant with a limited function. In one embodiment, treatment of a subject with a variant having a subset of the biological activities of the naturally occurring form of the reagent has fewer side effects in a subject relative to treatment with the naturally occurring form of the reagent.

Preferably, the analog, variant, or derivative reagent is functionally active. As utilized herein, the term “functionally active” refers to species displaying one or more known functional attributes of a full-length reagent. “Variant” refers to a reagent differing from naturally occurring reagent, but retaining essential properties thereof. Generally, variants are overall closely similar, and in many regions, identical to the naturally occurring reagent.

Variants of the reagent that function as either agonists (mimetics) or as antagonists can be identified by screening combinatorial libraries of mutants of the reagent for peptide agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential sequences is expressible as individual peptides, or alternatively, as a set of larger fusion proteins (e.g., for phage display) containing the set of sequences therein. There are a variety of methods which can be used to produce libraries of potential variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential sequences. Methods for synthesizing degenerate oligonucleotides are known in the art (see, e.g., Narang (1983) Tetrahedron 39:3; Itakura et al. (1984) Annu Rev Biochem 53:323; Itakura et al. (1984) Science 198:1056; Ike et al. (1983) Nucl. Acids Res. 11:477.

Derivatives and analogs of the reagent or individual moieties can be produced by various methods known within the art. For example, the polypeptide sequences may be modified by any number of methods known within the art. See e.g., Sambrook, et al., 1990. Molecular Cloning: A Laboratory Manual, 2nd ed., (Cold Spring Harbor Laboratory Press; Cold Spring Harbor, N.Y.). Modifications include: glycosylation, acetylation, phosphorylation, amidation, derivatization by known protecting/blocking groups, linkage to an antibody molecule or other cellular reagent, and the like. Any of the numerous chemical modification methodologies known within the art may be utilized including, but not limited to, specific chemical cleavage by cyanogen bromide, trypsin, chymotrypsin, papain, V8 protease, NaBH₄, acetylation, formylation, oxidation, reduction, metabolic synthesis in the presence of tunicamycin, etc.

Derivatives and analogs may be full length or other than full length, if said derivative or analog contains a modified nucleic acid or amino acid, as described infra. Derivatives or analogs of the reagent include, but are not limited to, molecules comprising regions that are substantially homologous in various embodiments, of at least 30%, 40%, 50%, 60%, 70%, 80%, 90% or preferably 95% amino acid identity when: (i) compared to an amino acid sequence of identical size; (ii) compared to an aligned sequence in that the alignment is done by a computer homology program known within the art (e.g., Wisconsin GCG software) or (iii) the encoding nucleic acid is capable of hybridizing to a sequence encoding the aforementioned peptides under stringent (preferred), moderately stringent, or non-stringent conditions. See, e.g., Ausubel, et al., Current Protocols in Molecular Biology, John Wiley and Sons, New York, N.Y., 1993.

Derivatives of the reagent may be produced by alteration of their sequences by substitutions, additions or deletions that result in functionally-equivalent molecules. One or more amino acid residues within the reagent may be substituted by another amino acid of a similar polarity and net charge, thus resulting in a silent alteration. Conservative substitutes for an amino acid within the sequence may be selected from other members of the class to which the amino acid belongs. For example, nonpolar (hydrophobic) amino acids include alanine, leucine, isoleucine, valine, proline, phenylalanine, tryptophan and methionine. Polar neutral amino acids include glycine, serine, threonine, cysteine, tyrosine, asparagine, and glutamine. Positively charged (basic) amino acids include arginine, lysine and histidine. Negatively charged (acidic) amino acids include aspartic acid and glutamic acid.

The reagent can be administered locally to any loci implicated in the CNS disorder pathology, i.e. any loci deficient in neural cells as a cause of the disease. For example, the reagent can be administered locally to the ventricle of the brain, substantia nigra, striatum, locus ceruleous, nucleus basalis Meynert, pedunculopontine nucleus, cerebral cortex, and spinal cord.

Neural stem cells and their progeny can be induced to proliferate and differentiate in vivo by administering to the host a reagent, alone or in combination with other agents, or by administering a pharmaceutical composition containing the reagent that will induce proliferation and differentiation of the cells. Pharmaceutical compositions include any substance that blocks the inhibitory influence and/or stimulates neural stem cells and stem cell progeny to proliferate and ultimately differentiate. Such in vivo manipulation and modification of these cells allows cells lost, due to injury or disease, to be endogenously replaced, thus obviating the need for transplanting foreign cells into a patient.

Antibodies

Included in the invention are antibodies to be used as reagents. The term “antibody” as used herein refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab′} and F_{(ab′)2} fragments, and an F_{ab} expression library. In general, antibody molecules obtained from humans relates to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain. Reference herein to antibodies includes a reference to all such classes, subclasses and types of human antibody species.

An isolated protein of the invention intended to serve as an antigen, or a portion or fragment thereof, can be used as an immunogen to generate antibodies that immunospecifically bind the antigen, using standard techniques for polyclonal and monoclonal antibody preparation. The full-length protein can be used or, alternatively, the invention provides antigenic peptide fragments of the antigen for use as immunogens. An antigenic peptide fragment comprises at least 6 amino acid residues of the amino acid sequence of the full length protein and encompasses an epitope thereof such that an antibody raised against the peptide forms a specific immune complex with the full length protein or with any fragment that contains the epitope. Preferably, the antigenic peptide comprises at least 10 amino acid residues, or at least 15 amino acid residues, or at least 20 amino acid residues, or at least 30 amino acid residues. Preferred epitopes encompassed by the antigenic peptide are regions of the protein that are located on its surface; commonly these are hydrophilic regions.

In certain embodiments of the invention, at least one epitope encompassed by the antigenic peptide is a region of EphA7, ephrin-A5 or ephrin-A2 that is located on the surface of the protein, e.g., a hydrophilic region. A hydrophobicity analysis of the human those protein sequences will indicate which regions of the polypeptide are particularly hydrophilic and, therefore, are likely to encode surface residues useful for targeting antibody production. As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation. See, e.g., Hopp and Woods, 1981, Proc. Nat. Acad. Sci. USA 78: 3824-3828; Kyte and Doolittle 1982, J. Mol. Biol. 157: 105-142, each incorporated herein by reference in their entirety. Antibodies that are specific for one or more domains within an antigenic protein, or derivatives, fragments, analogs or homologs thereof, are also provided herein.

The term “epitope” includes any protein determinant capable of specific binding to an immunoglobulin or T-cell receptor. Epitopic determinants usually consist of chemically active surface groupings of molecules such as amino acids or sugar side chains and usually have specific three dimensional structural characteristics, as well as specific charge characteristics. A EphA7, ephrin-A5 or ephrin-A2, or a fragment thereof comprises at least one antigenic epitope. An anti-EphA7, ephrin-A5 or ephrin-A2 antibody of the present invention is said to specifically bind to the antigen when the equilibrium binding constant (K_{D}) is ≦1 μM, preferably ≦100 nM, more preferably ≦10 nM, and most preferably ≦100 pM to about 1 pM, as measured by assays such as radioligand binding assays or similar assays known to those skilled in the art.

Various procedures known within the art may be used for the production of polyclonal or monoclonal antibodies directed against a protein of the invention, or against derivatives, fragments, analogs homologs or orthologs thereof (see, for example, Antibodies: A Laboratory Manual, Harlow E, and Lane D, 1988, Cold Spring Harbor Laboratory Press, Cold Spring Harbor, N.Y., incorporated herein by reference). Some of these antibodies are discussed below.

Polyclonal Antibodies

For the production of polyclonal antibodies, various suitable host animals (e.g., rabbit, goat, mouse or other mammal) may be immunized by one or more injections with the native protein, a synthetic variant thereof, or a derivative of the foregoing. An appropriate immunogenic preparation can contain, for example, the naturally occurring immunogenic protein, a chemically synthesized polypeptide representing the immunogenic protein, or a recombinantly expressed immunogenic protein. Furthermore, the protein may be conjugated to a second protein known to be immunogenic in the mammal being immunized. Examples of such immunogenic proteins include but are not limited to keyhole limpet hemocyanin, serum albumin, bovine thyroglobulin, and soybean trypsin inhibitor. The preparation can further include an adjuvant. Various adjuvants used to increase the immunological response include, but are not limited to, Freund's (complete and incomplete), mineral gels (e.g., aluminum hydroxide), surface active substances (e.g., lysolecithin, pluronic polyols, polyanions, peptides, oil emulsions, dinitrophenol, etc.), adjuvants usable in humans such as Bacille Calmette-Guerin and Corynebacterium parvum, or similar immunostimulatory agents. Additional examples of adjuvants which can be employed include MPL-TDM adjuvant (monophosphoryl Lipid A, synthetic trehalose dicorynomycolate).

The polyclonal antibody molecules directed against the immunogenic protein can be isolated from the mammal (e.g., from the blood) and further purified by well known techniques, such as affinity chromatography using protein A or protein G, which provide primarily the IgG fraction of immune serum. Subsequently, or alternatively, the specific antigen which is the target of the immunoglobulin sought, or an epitope thereof, may be immobilized on a column to purify the immune specific antibody by immunoaffinity chromatography. Purification of immunoglobulins is discussed, for example, by D. Wilkinson (The Scientist, published by The Scientist, Inc., Philadelphia Pa., Vol. 14, No. 8 (Apr. 17, 2000), pp. 25-28).

Monoclonal Antibodies

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs thus contain an antigen binding site capable of immunoreacting with a particular epitope of the antigen characterized by a unique binding affinity for it.

Monoclonal antibodies can be prepared using hybridoma methods, such as those described by Kohler and Milstein, Nature, 256:495 (1975). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes can be immunized in vitro.

The immunizing agent will typically include the protein antigen, a fragment thereof or a fusion protein thereof. Generally, either peripheral blood lymphocytes are used if cells of human origin are desired, or spleen cells or lymph node cells are used if non-human mammalian sources are desired. The lymphocytes are then fused with an immortalized cell line using a suitable fusing agent, such as polyethylene glycol, to form a hybridoma cell (Goding, Monoclonal Antibodies: Principles and Practice, Academic Press, (1986) pp. 59-103). Immortalized cell lines are usually transformed mammalian cells, particularly myeloma cells of rodent, bovine and human origin. Usually, rat or mouse myeloma cell lines are employed. The hybridoma cells can be cultured in a suitable culture medium that preferably contains one or more substances that inhibit the growth or survival of the unfused, immortalized cells. For example, if the parental cells lack the enzyme hypoxanthine guanine phosphoribosyl transferase (HGPRT or HPRT), the culture medium for the hybridomas typically will include hypoxanthine, aminopterin, and thymidine (“HAT medium”), which substances prevent the growth of HGPRT-deficient cells.

Preferred immortalized cell lines are those that fuse efficiently, support stable high level expression of antibody by the selected antibody-producing cells, and are sensitive to a medium such as HAT medium. More preferred immortalized cell lines are murine myeloma lines, which can be obtained, for instance, from the Salk Institute Cell Distribution Center, San Diego, Calif. and the American Type Culture Collection, Manassas, Va. Human myeloma and mouse-human heteromyeloma cell lines also have been described for the production of human monoclonal antibodies (Kozbor, J. Immunol., 133:3001 (1984); Brodeur et al., Monoclonal Antibody Production Techniques and Applications, Marcel Dekker, Inc., New York, (1987) pp. 51-63).

The culture medium in which the hybridoma cells are cultured can then be assayed for the presence of monoclonal antibodies directed against the antigen. Preferably, the binding specificity of monoclonal antibodies produced by the hybridoma cells is determined by immunoprecipitation or by an in vitro binding assay, such as radioimmunoassay (RIA) or enzyme-linked immunoabsorbent assay (ELISA). Such techniques and assays are known in the art. The binding affinity of the monoclonal antibody can, for example, be determined by the Scatchard analysis of Munson and Pollard, Anal. Biochem., 107:220 (1980). It is an objective, especially important in therapeutic applications of monoclonal antibodies, to identify antibodies having a high degree of specificity and a high binding affinity for the target antigen.

After the desired hybridoma cells are identified, the clones can be subcloned by limiting dilution procedures and grown by standard methods (Goding, Monoclonal antibodies: principles and practice, Academic press, (1986) pp. 59-103). Suitable culture media for this purpose include, for example, Dulbecco's Modified Eagle's Medium and RPMI-1640 medium. Alternatively, the hybridoma cells can be grown in vivo as ascites in a mammal.

The monoclonal antibodies secreted by the subclones can be isolated or purified from the culture medium or ascites fluid by conventional immunoglobulin purification procedures such as, for example, protein A-Sepharose, hydroxylapatite chromatography, gel electrophoresis, dialysis, or affinity chromatography.

The monoclonal antibodies can also be made by recombinant DNA methods, such as those described in U.S. Pat. No. 4,816,567. DNA encoding the monoclonal antibodies of the invention can be readily isolated and sequenced using conventional procedures (e.g., by using oligonucleotide probes that are capable of binding specifically to genes encoding the heavy and light chains of murine antibodies). The hybridoma cells of the invention serve as a preferred source of such DNA. Once isolated, the DNA can be placed into expression vectors, which are then transfected into host cells such as simian COS cells, Chinese hamster ovary (CHO) cells, or myeloma cells that do not otherwise produce immunoglobulin protein, to obtain the synthesis of monoclonal antibodies in the recombinant host cells. The DNA also can be modified, for example, by substituting the coding sequence for human heavy and light chain constant domains in place of the homologous murine sequences (U.S. Pat. No. 4,816,567; Morrison, Nature 368, 812-13 (1994)) or by covalently joining to the immunoglobulin coding sequence all or part of the coding sequence for a non-immunoglobulin polypeptide. Such a non-immunoglobulin polypeptide can be substituted for the constant domains of an antibody of the invention, or can be substituted for the variable domains of one antigen-combining site of an antibody of the invention to create a chimeric bivalent antibody.

Humanized Antibodies

The antibodies directed against the protein antigens of the invention can further comprise humanized antibodies or human antibodies. These antibodies are suitable for administration to humans without engendering an immune response by the human against the administered immunoglobulin. Humanized forms of antibodies are chimeric immunoglobulins, immunoglobulin chains or fragments thereof (such as Fv, Fab, Fab′, F(ab′)₂ or other antigen-binding subsequences of antibodies) that are principally comprised of the sequence of a human immunoglobulin and contain minimal sequence derived from a non-human immunoglobulin. Humanization can be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody. (See also U.S. Pat. No. 5,225,539.) In some instances, Fv framework residues of the human immunoglobulin are replaced by corresponding non-human residues. Humanized antibodies can also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin (Jones et al., 1986; Riechmann et al., 1988; and Presta, Curr. Op. Struct. Biol., 2:593-596 (1992)).

Human Antibodies

Fully human antibodies essentially relate to antibody molecules in which the entire sequence of both the light chain and the heavy chain, including the CDRs, arise from human genes. Such antibodies are termed “human antibodies”, or “fully human antibodies” herein. Human monoclonal antibodies can be prepared by the trioma technique; the human B-cell hybridoma technique (see Kozbor, et al., 1983 Immunol Today 4: 72) and the EBV hybridoma technique to produce human monoclonal antibodies (see Cole, et al., 1985 In: MONOCLONAL ANTIBODIES AND CANCER THERAPY, Alan R. Liss, Inc., pp. 77-96). Human monoclonal antibodies may be utilized in the practice of the present invention and may be produced by using human hybridomas (see Cote, et al., 1983. Proc Natl Acad Sci USA 80: 2026-2030) or by transforming human B-cells with Epstein Barr Virus in vitro (see Cole, et al., 1985 In: MONOCLONAL ANTIBODIES AND CANCER THERAPY, Alan R. Liss, Inc., pp. 77-96).

In addition, human antibodies can also be produced using additional techniques, including phage display libraries (Hoogenboom and Winter, J. Mol. Biol., 227:381 (1991); Marks et al., J. Mol. Biol., 222:581 (1991)). Similarly, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in Marks et al. (Bio/Technology 10, 779-783 (1992)); Lonberg et al. (Nature 368 856-859 (1994)); Morrison (Nature 368, 812-13 (1994)); Fishwild et al, (Nature Biotechnology 14, 845-51 (1996)); Neuberger (Nature Biotechnology 14, 826 (1996)); and Lonberg and Huszar (Intern. Rev. Immunol. 13 65-93 (1995)).

Human antibodies may additionally be produced using transgenic nonhuman animals which are modified so as to produce fully human antibodies rather than the animal's endogenous antibodies in response to challenge by an antigen. (See PCT publication WO94/02602). The endogenous genes encoding the heavy and light immunoglobulin chains in the nonhuman host have been incapacitated, and active loci encoding human heavy and light chain immunoglobulins are inserted into the host's genome. The human genes are incorporated, for example, using yeast artificial chromosomes containing the requisite human DNA segments. An animal which provides all the desired modifications is then obtained as progeny by crossbreeding intermediate transgenic animals containing fewer than the full complement of the modifications. The preferred embodiment of such a nonhuman animal is a mouse, and is termed the Xenomouse™ as disclosed in PCT publications WO 96/33735 and WO 96/34096. This animal produces B cells which secrete fully human immunoglobulins. The antibodies can be obtained directly from the animal after immunization with an immunogen of interest, as, for example, a preparation of a polyclonal antibody, or alternatively from immortalized B cells derived from the animal, such as hybridomas producing monoclonal antibodies. Additionally, the genes encoding the immunoglobulins with human variable regions can be recovered and expressed to obtain the antibodies directly, or can be further modified to obtain analogs of antibodies such as, for example, single chain Fv molecules.

An example of a method of producing a nonhuman host, exemplified as a mouse, lacking expression of an endogenous immunoglobulin heavy chain is disclosed in U.S. Pat. No. 5,939,598. It can be obtained by a method including deleting the J segment genes from at least one endogenous heavy chain locus in an embryonic stem cell to prevent rearrangement of the locus and to prevent formation of a transcript of a rearranged immunoglobulin heavy chain locus, the deletion being effected by a targeting vector containing a gene encoding a selectable marker; and producing from the embryonic stem cell a transgenic mouse whose somatic and germ cells contain the gene encoding the selectable marker.

A method for producing an antibody of interest, such as a human antibody, is disclosed in U.S. Pat. No. 5,916,771. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

In a further improvement on this procedure, a method for identifying a clinically relevant epitope on an immunogen, and a correlative method for selecting an antibody that binds immunospecifically to the relevant epitope with high affinity, are disclosed in PCT publication WO 99/53049.

F_{ab} Fragments and Single Chain Antibodies

According to the invention, techniques can be adapted for the production of single-chain antibodies specific to an antigenic protein of the invention (see e.g., U.S. Pat. No. 4,946,778). In addition, methods can be adapted for the construction of F_{ab} expression libraries (see e.g., Huse, et al., 1989 Science 246: 1275-1281) to allow rapid and effective identification of monoclonal F_{ab} fragments with the desired specificity for a protein or derivatives, fragments, analogs or homologs thereof. Antibody fragments that contain the idiotypes to a protein antigen may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent and (iv) Fᵥ fragments.

Bispecific Antibodies

Bispecific antibodies are monoclonal, preferably human or humanized, antibodies that have binding specificities for at least two different antigens. In the present case, one of the binding specificities is for an antigenic protein of the invention. The second binding target is any other antigen, and advantageously is a cell-surface protein or receptor or receptor subunit.

Methods for making bispecific antibodies are known in the art. Traditionally, the recombinant production of bispecific antibodies is based on the co-expression of two immunoglobulin heavy-chain/light-chain pairs, where the two heavy chains have different specificities (Milstein and Cuello, Nature, 305:537-539 (1983)). Because of the random assortment of immunoglobulin heavy and light chains, these hybridomas (quadromas) produce a potential mixture of ten different antibody molecules, of which only one has the correct bispecific structure. The purification of the correct molecule is usually accomplished by affinity chromatography steps. Similar procedures are disclosed in WO 93/08829, published 13 May 1993, and in Traunecker et al., EMBO J., 10:3655-3659 (1991).

Antibody variable domains with the desired binding specificities (antibody-antigen combining sites) can be fused to immunoglobulin constant domain sequences. The fusion preferably is with an immunoglobulin heavy-chain constant domain, comprising at least part of the hinge, CH2, and CH3 regions. It is preferred to have the first heavy-chain constant region (CH1) containing the site necessary for light-chain binding present in at least one of the fusions. DNAs encoding the immunoglobulin heavy-chain fusions and, if desired, the immunoglobulin light chain, are inserted into separate expression vectors, and are co-transfected into a suitable host organism. For further details of generating bispecific antibodies see, for example, Suresh et al., Methods in Enzymology, 121:210 (1986).

According to another approach described in WO 96/27011, the interface between a pair of antibody molecules can be engineered to maximize the percentage of heterodimers which are recovered from recombinant cell culture. The preferred interface comprises at least a part of the CH3 region of an antibody constant domain. In this method, one or more small amino acid side chains from the interface of the first antibody molecule are replaced with larger side chains (e.g. tyrosine or tryptophan). Compensatory “cavities” of identical or similar size to the large side chain(s) are created on the interface of the second antibody molecule by replacing large amino acid side chains with smaller ones (e.g. alanine or threonine). This provides a mechanism for increasing the yield of the heterodimer over other unwanted end-products such as homodimers.

Bispecific antibodies can be prepared as full length antibodies or antibody fragments (e.g. F(ab′)₂ bispecific antibodies). Techniques for generating bispecific antibodies from antibody fragments have been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent sodium arsenite to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Additionally, Fab′ fragments can be directly recovered from E. coli and chemically coupled to form bispecific antibodies. Shalaby et al., J. Exp. Med. 175:217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a heavy-chain variable domain (V_{H}) connected to a light-chain variable domain (V_{L}) by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See, Gruber et al., J. Immunol. 152:5368 (1994). Antibodies with more than two valencies are contemplated. For example, trispecific antibodies can be prepared. Tutt et al., J. Immunol. 147:60 (1991).

Exemplary bispecific antibodies can bind to two different epitopes, at least one of which originates in the protein antigen of the invention. Alternatively, an anti-antigenic arm of an immunoglobulin molecule can be combined with an arm which binds to a triggering molecule on a leukocyte such as a T-cell receptor molecule (e.g. CD2, CD3, CD28, or B7), or Fc receptors for IgG (FcγR), such as FcγRI (CD64), FcγRII (CD32) and FcγRIII (CD16) so as to focus cellular defense mechanisms to the cell expressing the particular antigen. Bispecific antibodies can also be used to direct cytotoxic agents to cells which express a particular antigen. These antibodies possess an antigen-binding arm and an arm which binds a cytotoxic agent or a radionuclide chelator, such as EOTUBE, DPTA, DOTA or TETA. Another bispecific antibody of interest binds the protein antigen described herein and further binds tissue factor (TF).

Immunoliposomes

The antibodies disclosed herein can also be formulated as immunoliposomes. Liposomes containing the antibody are prepared by methods known in the art, such as described in Epstein et al., Proc. Natl. Acad. Sci. USA, 82: 3688 (1985); Hwang et al., Proc. Natl. Acad. Sci. USA, 77: 4030 (1980); and U.S. Pat. Nos. 4,485,045 and 4,544,545. Liposomes with enhanced circulation time are disclosed in U.S. Pat. No. 5,013,556.

Particularly useful liposomes can be generated by the reverse-phase evaporation method with a lipid composition comprising phosphatidylcholine, cholesterol, and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem., 257: 286-288 (1982) via a disulfide-interchange reaction.

Antibody Therapeutics

Antibodies of the invention, including polyclonal, monoclonal, humanized and fully human antibodies, may used as therapeutic agents such as one of this invention. Such agents will generally be employed to treat or prevent a disease or pathology, specifically neurological disease, in a subject. An antibody preparation, preferably one having high specificity and high affinity for its target antigen, is administered to the subject and will generally have an effect due to its binding with the target. Such an effect may be one of two kinds, depending on the specific nature of the interaction between the given antibody molecule and the target antigen in question. In the first instance, administration of the antibody may abrogate or inhibit the binding of the target with an endogenous EphA7, ephrin-A5 or ephrin-A2 ligand to which it naturally binds. In this case, the antibody binds to the target and masks a binding site of the naturally occurring ligand, wherein the ligand serves as an effector molecule. Thus, the receptor mediates a signal transduction pathway for which ligand is responsible.

Alternatively, the effect may be one in which the antibody elicits a physiological result by virtue of binding to an effector binding site on the target molecule. In this case the target, a EphA7, ephrin-A5 or ephrin-A2 cell surface receptor having an endogenous ligand which needs to be modulated, binds the antibody as a surrogate effector ligand, initiating a receptor-based signal transduction event by the receptor.

A therapeutically effective amount of an antibody of the invention relates generally to the amount needed to achieve a therapeutic objective. As noted above, this may be a binding interaction between the antibody and its target antigen that, in certain cases, interferes with the functioning of the target, and in other cases, promotes a physiological response. The amount required to be administered will furthermore depend on the binding affinity of the antibody for its specific antigen and the rate at which an administered antibody is depleted from the free volume of the subject to which it is administered.

Diseases and Disorders

Diseases and disorders that are characterized by altered (relative to a subject not suffering from the disease or disorder) levels or biological activity may be treated with therapeutics that antagonize (i.e., reduce or inhibit) EphA7, ephrin-A5 or ephrin-A2 activity. Therapeutics that antagonize activity may be administered in a therapeutic or prophylactic manner. Therapeutics that may be utilized include, but are not limited to: (i) an aforementioned peptide, analog, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i.e., due to a heterologous insertion within the coding sequences of coding sequences to an aforementioned peptide) that are utilized to “knockout” endogenous function of an aforementioned peptide by homologous recombination (see, e.g., Capecchi, 1989. Science 244:1288-1292); or (v) modulators (i.e., inhibitors, agonists and antagonists, including additional peptide mimetic of the invention or antibodies specific to a peptide of the invention) that alter the interaction between an aforementioned peptide and its binding partner.

Diseases and disorders that are characterized by altered (relative to a subject not suffering from the disease or disorder) levels or biological activity may be treated with therapeutics that increase (i.e., are agonists to) activity. Therapeutics that upregulate activity may be administered in a therapeutic or prophylactic manner. Therapeutics that may be utilized include, but are not limited to, an aforementioned peptide, analog, derivatives, fragments or homologs thereof, or an agonist that increases bioavailability.

Increased or decreased levels can be detected by quantifying peptide and/or RNA, by obtaining a patient tissue sample (e.g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e.g., by Western blot analysis, immunoprecipitation followed by sodium dodecyl sulfate (SDS) polyacrylamide gel electrophoresis, immunocytochemistry, etc.) and/or hybridization assays to detect expression of mRNAs (e.g., Northern assays, dot blots, in situ hybridization, and the like).

Therapeutic Methods

Another aspect of the invention pertains to methods of modulating EphA7, ephrin-A5 or ephrin-A2 expression or activity for therapeutic purposes. The modulatory method of the invention involves contacting a cell with an agent that modulates one or more of the activities of EphA7, ephrin-A5 or ephrin-A2 protein activity associated with the cell. An agent that modulates this protein activity can be an agent as described herein, such as a nucleic acid or a protein, a naturally-occurring cognate ligand of a EphA7, ephrin-A5 or ephrin-A2 receptor, a peptide, a EphA7, ephrin-A5 or ephrin-A2 peptidomimetic, or other small molecule. In one embodiment, the agent stimulates the activity of the EphA7, ephrin-A5 or ephrin-A2 signaling pathway. Examples of such stimulatory agents include active EphA7, ephrin-A5 or ephrin-A2 protein and a nucleic acid molecule encoding EphA7, ephrin-A5 or ephrin-A2 that has been introduced into the cell. In another embodiment, the agent inhibits EphA7, ephrin-A5 or ephrin-A2 signaling. Examples of such inhibitory agents include antisense nucleic acid molecules and antibodies. These modulatory methods can be performed in vitro (e.g., by culturing the cell with the agent) or, alternatively, in vivo (e.g., by administering the agent to a subject). As such, the invention provides methods of treating an individual afflicted with a disease or disorder, specifically a neurological disorder. In one embodiment, the method involves administering an reagent (e.g., an reagent identified by a screening assay described herein), or combination of reagents that modulate (e.g., up-regulates or down-regulates) EphA7, ephrin-A5 or ephrin-A2 expression or activity. In another embodiment, the method involves administering a EphA7, ephrin-A5 or ephrin-A2 protein or nucleic acid molecule as therapy to modulate proliferation, differentiation or survival of NSCs/NPCs.

Determination of the Biological Effect of the Therapeutic

In various embodiments of the invention, suitable in vitro or in vivo assays are performed to determine the effect of a specific therapeutic and whether its administration is indicated for treatment of the affected tissue.

In various specific embodiments, in vitro assays may be performed with representative stem cells or newly differentiated cells involved in the patient's disorder, to determine if a given therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

Pharmaceutical Compositions

The invention provides methods of influencing central nervous system cells to produce progeny that can replace damaged or missing neurons in the central nervous system by exposing a patient, suffering from a neurological disease or disorder, to a reagent (e.g. EphA7, ephrin-A5 or ephrin-A2) in a suitable formulation through a suitable route of administration, that modulates NSC or NPC activity in vivo. A “neurological disease or disorder” is a disease or disorder which results in the disturbance in the structure or function of the central nervous system resulting from developmental abnormality, disease, injury or toxin. Examples of neurological diseases or disorders include neurodegenerative disorders (e.g. associated with Parkinson's disease, Alzheimer's disease, Huntington's disease, Shy-Drager Syndrome, Progressive Supranuclear Palsy, Lewy Body Disease or Amyotrophic Lateral Sclerosis); ischemic disorders (e.g. cerebral or spinal cord infarction and ischemia, stroke); traumas (e.g. caused by physical injury or surgery, and compression injuries; affective disorders (e.g. stress, depression and post-traumatic depression); neuropsychiatric disorders (e.g. schizophrenia, multiple sclerosis or epilepsy); and learning and memory disorders.

This invention provides a method of treating a neurological disease or disorder comprising administering a reagent that modulates neural stem cell or neural progenitor cell activity in vivo to a mammal. The term “mammal” refers to any mammal classified as a mammal, including humans, cows, horses, dogs, sheep and cats. In one embodiment, the mammal is a human.

The invention provides a regenerative cure for neurodegenerative diseases by stimulating ependymal cells and subventricular zone cells to proliferate, migrate and differentiate into the desired neural phenotype targeting loci where cells are damaged or missing. In vivo stimulation of ependymal stem cells is accomplished by locally administering a reagent to the cells in an appropriate formulation. By increasing neurogenesis, damaged or missing neurons can be replaced in order to enhance brain function in diseased states.

A pharmaceutical composition useful as a therapeutic agent for the treatment of central nervous system disorders is provided. For example, the composition includes a reagent of the invention, which can be administered alone or in combination with the systemic or local co-administration of one or more additional agents. Such agents include preservatives, ventricle wall permeability increasing factors, stem cell mitogens, survival factors, glial lineage preventing agents, anti-apoptotic agents, anti-stress medications, neuroprotectants, and anti-pyrogenics. The pharmaceutical composition preferentially treats CNS diseases by stimulating cells (e.g., ependymal cells and subventricular zone cells) to proliferate, migrate and differentiate into the desired neural phenotype, targeting loci where cells are damaged or missing.

A method for treating a subject suffering from a CNS disease or disorder is also provided. This method comprises administering to the subject an effective amount of a pharmaceutical composition containing a reagent (1) alone in a dosage range of 0.5 ng/kg/day to 500 ng/kg/day, (2) in a combination with a ventricle wall permeability increasing factor, or (3) in combination with a locally or systemically co-administered agent.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., chimeric peptide) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipients such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Nucleic acid molecules encoding a proteinaceous agent can be inserted into vectors and used as gene therapy vectors. Gene therapy vectors can be delivered to a subject by, for example, intravenous injection, local administration (see U.S. Pat. No. 5,328,470) or by stereotactic injection (see e.g., Chen et al. (1994) PNAS 91:3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells that produce the gene delivery system.

The pharmaceutical compositions can be included in a container, pack, or dispenser together with instructions for administration.

In another embodiments, the reagent is administered in a composition comprising at least 90% pure reagent. The reagent can be, for example EphA7, ephrin-A5 or ephrin-A2 or a EphA7, ephrin-A5 or ephrin-A2 receptor, or any combination thereof.

Preferably the reagent is formulated in a medium providing maximum stability and the least formulation-related side-effects. In addition to the reagent, the composition of the invention will typically include one or more protein carrier, buffer, isotonic salt and stabilizer.

In some instances, the reagent can be administered by a surgical procedure implanting a catheter coupled to a pump device. The pump device can also be implanted or be extracorporally positioned. Administration of the reagent can be in intermittent pulses or as a continuous infusion. Devices for injection to discrete areas of the brain are known in the art (see, e.g., U.S. Pat. Nos. 6,042,579; 5,832,932; and 4,692,147).

Reagents containing compositions can be administered in any conventional form for administration of a protein. A reagent can be administered in any manner known in the art in which it may either pass through or by-pass the blood-brain barrier. Methods for allowing factors to pass through the blood-brain barrier include minimizing the size of the factor, providing hydrophobic factors which may pass through more easily, conjugating the protein reagent or other agent to a carrier molecule that has a substantial permeability coefficient across the blood brain barrier (see, e.g., U.S. Pat. No. 5,670,477).

Reagents, derivatives, and co-administered agents can be incorporated into pharmaceutical compositions suitable for administration. Such compositions typically comprise the agent and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions. Modifications can be made to the agents to affect solubility or clearance of the peptide. Peptidic molecules may also be synthesized with D-amino acids to increase resistance to enzymatic degradation. In some cases, the composition can be co-administered with one or more solubilizing agents, preservatives, and permeation enhancing agents.

For example, the composition can include a preservative or a carrier such as proteins, carbohydrates, and compounds to increase the density of the pharmaceutical composition. The composition can also include isotonic salts and redox-control agents.

In some embodiments, the composition administered includes the reagent and one or more agents that increase the permeability of the ventricle wall, i.e. “ventricle wall permeability enhancers.” Such a composition can help an injected composition penetrate deeper than the ventricle wall. Examples of suitable ventricle wall permeability enhancers include, for example, liposomes, VEGF (vascular endothelial growth factor), IL-s, TNFα, polyoxyethylene, polyoxyethylene ethers of fatty acids, sorbitan monooleate, sorbitan monolaurate, polyoxyethylene monolaurate, polyoxyethylene sorbitan monolaurate, fusidic acid and derivatives thereof, EDTA, disodium EDTA, cholic acid and derivatives, deoxycholic acid, glycocholic acid, glycodeoxycholic acid, taurocholic acid, taurodeoxycholic acid, sodium cholate, sodium glycocholate, glycocholate, sodium deoxycholate, sodium taurocholate, sodium glycodeoxycholate, sodium taurodeoxycholate, chenodeoxycholic acid, urosdeoxycholic acid, saponins, glycyrrhizic acid, ammonium glycyrrhizide, decamethonium, decamethonium bromide, dodecyltrimethylammonium bromide, and dimethyl-β-cyclodextrin or other cyclodextrins.

Drug Screening

The invention also provide a method of using the receptors or receptor/reagent complexes for analyzing or purifying certain stem or progenitor cell populations, using e.g. antibodies, against the receptors or receptor/reagent complexes.

In another aspect, the invention provides a method for screening for reagents that influence stem and progenitor cells. In some applications, neural cells (undifferentiated or differentiated) are used to screen factors that promote maturation into neural cells, or promote proliferation and maintenance of such cells in long-term culture. For example, candidate reagents are tested by adding them to cells in culture at varying dosages, and then determining any changes that result, according to desirable criteria for further culture and use of the cells. Physical characteristics of the cells can be analyzed by observing cell and neurite growth with microscopy. The induction of expression of increased levels of proliferation, differentiation and migration can be analyzed with any technique known in the art which can identify proliferation and differentiation. Such techniques include RT-PCR, in situ hybridisation, and ELISA.

In one aspect, novel receptor/reagents in undifferentiated neurospheres was examined using RT-PCR techniques. In particular, genes that are up-regulated in these undifferentiated neurospheres were identified. As used herein, the term “up-regulation” refers to a process that increases reagent/receptor interactions due to an increase in the number of available receptors. The presence of these genes suggests a potential role in the mediation of signal transduction pathways in the regulation of NSC/NPC function. Furthermore, by knowing the levels of expression of the receptors or their various reagents, it is possible to diagnose disease or determine the role of stem and progenitor cells in the disease. By analyzing the genetic or amino-acid sequence variations in these genes or gene products, it is possible to diagnose or predict the development of certain diseases. Such analysis will provide the necessary information to determine the usefulness of using stem or progenitor cell based treatments for disease.

In another aspect, in situ hybridization is performed on adult mouse brain sections to determine which cells in the adult brain express these signalling pathways. This data is helpful in determining treatment options for various neurological diseases.

In yet another aspect, quantitative PCR is performed on RNA prepared from undifferentiated and differentiated neurospheres. In some embodiments, certain receptor-reagent combinations reveal much higher expression in the undifferentiated neurospheres as compared to neurospheres that have been induced to differentiate, while in other embodiments, other receptor-reagent combinations reveal the opposite. Undifferentiated neurospheres (which are rapidly proliferating cells with the capacity to differentiate into neurons and glial cells, which express higher levels of these receptor-reagent combinations) are involved in the pathways of proliferation and differentiation of NSC/NPC. For certain signalling pathways, the data indicating that they are expressed more in differentiated neurospheres suggests a role for this receptor-reagent combination in cells embarking or proceeding on a differentiation pathway.

To determine the effect of a potential reagent on neural cells, a culture of NSCs/NPCs derived from multipotent stem cells can be obtained from normal neural tissue or, alternatively, from a host afflicted with a CNS disease or disorder. The choice of culture will depend upon the particular agent being tested and the effects one wishes to achieve. Once the cells are obtained from the desired donor tissue, they are proliferated in vitro in the presence of a proliferation-inducing reagent.

The ability of various biological agents to increase, decrease or modify in some other way the number and nature of the stem cell progeny proliferated in the presence of the proliferative factor can be screened on cells proliferated by the methods previously discussed. For example, it is possible to screen for reagents that increase or decrease the proliferative ability of NSCs/NPCs which would be useful for generating large numbers of cells for transplantable purposes. In these studies precursor cells are plated in the presence of the reagent in question and assayed for the degree of proliferation and survival or progenitor cells and their progeny can be determined. It is possible to screen neural cells which have already been induced to differentiate prior to the screening. It is also possible to determine the effects of the reagent on the differentiation process by applying them to precursors cells prior to differentiation. Generally, the reagent will be solubilized and added to the culture medium at varying concentrations to determine the effect of the agent at each dose. The culture medium may be replenished with the reagent every couple of days in amounts so as to keep the concentration of the reagent somewhat constant.

Changes in proliferation are observed by an increase or decrease in the number of neurospheres that form and/or an increase or decrease in the size of the neurospheres, which is a reflection of the rate of proliferation and is determined by the numbers of precursor cells per neurosphere.

Using these screening methods, it is possible to screen for potential drug side-effects on prenatal and postnatal CNS cells by testing for the effects of the biological agents on stem cell and progenitor cell proliferation and on progenitor cell differentiation or the survival and function of differentiated CNS cells.

Other screening applications of this invention relate to the testing of pharmaceutical compounds for their effect on neural tissue. Screening may be done either because the compound is designed to have a pharmacological effect on neural cells, or because a compound designed to have effects elsewhere may have unintended side effects on the nervous system. The screening can be conducted using any of the neural precursor cells or terminally differentiated cells of the invention.

Effect of cell function can be assessed using any standard assay to observe phenotype or activity of neural cells, such as receptor binding, proliferation, differentiation, survival-either in cell culture or in an appropriate model.

Therapeutic Uses

The fact that neural stem cells are located in the tissues lining ventricles of mature brains offers several advantages for the modification and manipulation of these cells in vivo and the ultimate treatment of various neurological diseases, disorders, and injury that affect different regions of the CNS. Therapy for these diseases can be tailored accordingly so that stem cells surrounding ventricles near the affected region would be manipulated or modified in vivo using the methods described herein. The ventricular system is found in nearly all brain regions and thus allows easier access to the affected areas. In order to modify the stem cells in vivo by exposing them to a composition comprising a reagent, it is relatively easy to implant a device that administers the composition to the ventricle and thus, to the neural stem cells. For example, a cannula attached to an osmotic pump may be used to deliver the composition. Alternatively, the composition may be injected directly into the ventricles. The neural stem cell progeny can migrate into regions that have been damaged as a result of injury or disease. Furthermore, the close proximity of the ventricles to many brain regions would allow for the diffusion of a secreted neurological agent by the stem cells or their progeny.

In an additional embodiment, a reagent of the invention is administered locally, as described above, in combination with an agent administered locally or systemically. Such agents include, for example, one or more stem cell mitogens, survival factors, glial-lineage preventing agents, anti-apoptotic agents, anti-stress medications, neuroprotectants, and anti-pyrogenics, or any combination thereof.

The agent is administered systemically before, during, or after administration of the reagent of the invention. The locally administered agent can be administered before, during, or after the reagent administration.

For treatment of Huntington's Disease, Alzheimer's Disease, Parkinson's Disease, and other neurological disorders affecting primarily the forebrain, a reagent alone or with an additional agent or agents is delivered to the ventricles of the forebrain to affect in vivo modification or manipulation of the stem cells. For example, Parkinson's Disease is the result of low levels of dopamine in the brain, particularly the striatum. It is therefore advantageous to induce a patient's own quiescent stem cells to begin to divide in vivo and to induce the progeny of these cells to differentiate into dopaminergic cells in the affected region of the striatum, thus locally raising the levels of dopamine.

Normally the cell bodies of dopaminergic neurons are located in the substantia nigra and adjacent regions of the mesencephalon, with the axons projecting to the striatum. The methods and compositions of the invention provide an alternative to the use of drugs and the controversial use of large quantities of embryonic tissue for treatment of Parkinson's disease. Dopamine cells can be generated in the striatum by the administration of a composition comprising a reagent of the invention to the lateral ventricle.

For the treatment of MS and other demyelinating or hypomyelinating disorders, and for the treatment of Amyotrophic Lateral Sclerosis or other motor neuron diseases, a reagent of the invention, alone or with an additional agent or agents is delivered to the central canal.

In addition to treating CNS tissue immediately surrounding a ventricle, a reagent of the invention, alone or with an additional agent or agents can be administered to the lumbar cistern for circulation throughout the CNS.

In other aspects, neuroprotectants can also be co-administered systemically or locally before, during and/or after infusion of a regent of the invention. Neuroprotectants include antioxidants (agents with reducing activity, e.g., selenium, vitamin E, vitamin C, glutathione, cysteine, flavinoids, quinolines, enzymes with reducing activity, etc), Ca-channel modulators, Na-channel modulators, glutamate receptor modulators, serotonin receptor agonists, phospholipids, unsaturated- and polyunsaturated fatty acids, estrogens and selective estrogen receptor modulators (SERMS), progestins, thyroid hormone and thyroid hormone-mimicking compounds, cyclosporin A and derivatives, thalidomide and derivatives, methylxanthines, MAO inhibitors; serotonin-, noradrenaline and dopamine uptake blockers; dopamine agonists, L-DOPA, nicotine and derivatives, and NO synthase modulators.

Certain reagents of the invention may be pyrogenic following IV injection (in rats; Am. J. Physiol. Regul. Integr. Comp. Physiol. 2000 278:R1275-81). Thus, in some aspects of the invention, antipyrogenic agents like cox2 inhibitors, indomethacin, salisylic acid derivatives and other general anti-inflammatory/anti-pyrogenic compounds can be systemically or locally administered before, during and/or after administration of the reagent of the invention.

In another aspect of the invention, anti-apoptotic agents including caspase inhibitors and agents useful for antisense-modulation of apoptotic enzymes and factors can be administered before, during, or after administration of the reagent of the invention.

Stress syndromes lower neurogenesis, therefore in some aspects, it may be desirable to treat a subject with anti-stress medications such as, e.g., anti-glucocorticoids (e.g., RU486) and beta-blockers, administered systemically or locally before, during and/or after infusion of the reagent of the invention.

Methods for preparing the reagent dosage forms are known, or will be apparent, to those skilled in this art.

The amount of reagent to be administered will depend upon the exact size and condition of the patient, but will be from 0.1 ng/kg/day to 10 mg ng/kg/day in a volume of 0.001 to 10 ml.

The duration of treatment and time period of administration of reagent will also vary according to the size and condition of the patient, the severity of the illness and the specific composition and method being used.

The effectiveness of each of the foregoing methods for treating a patient with a CNS disease or disorder is assessed by any known standardized test for evaluating the disease.

Other features of the invention will become apparent in the course of the following description of exemplary embodiments which are given for illustration of the invention and are not intended to be limiting thereof. All references, patents and patent applications cited are hereby incorporated by reference in their entirety.

### EXAMPLES

### Example 1

### EphA7-FL, EphA7-T1 and EphA7-T2 Expression in Normal and Mutant Animals

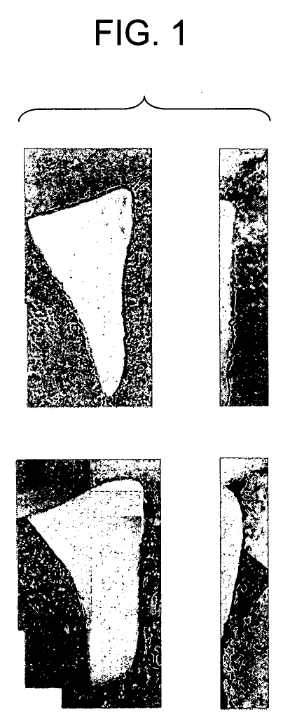

EphA7-FL, EphA7-T1 and EphA7-T2 are expressed in the neurogenic lateral wall of the lateral ventricle (See FIG. 1). EphA7^{-/-} mutants have small and narrow lateral ventricles due to increased amount of parenchymal tissue, indicating increased proliferation. The EphA7^{-/-} single mutant displays severely altered tissue architecture in the lateral ventricles. The tissue in the lateral side of the ventricle has expanded into the ventricular space, which efficiently narrows the lateral ventricle. When injected with BrdU to label dividing cells, adult EphA7^{-/-} mutant mice show significantly increased labelling in the neurogenic SVZ compared to wild type mice. Ephrin-A2, ephrin-A5 and ephA7 are expressed in neurospheres obtained from the lateral wall of the lateral ventricle.

When cultivated, the total yield of neurospheres obtained from ephA7^{-/-} mice is higher than the yield from wild type mice. Primary cultures obtained from the lateral ventricle's lateral wall of adult EphA7^{-/-} mutant mice contain a higher number of neurospheres than cultures obtained from wild type mice. The amount of neurospheres from EphA7^{-/-} being 1.33 times higher than spheres obtained from wild type animals (n=3).

Intracranial infusion of ephrin-A5-Fc increases the number of brdu positive cells in the anterior part of the lateral wall of the lateral ventricle. Osmotic pumps filled with ephrin-A5-Fc were allowed to deliver the proteins through intracranial infusion into the lateral ventricles of adult mice for 3.5 days. Intraperitoneal injections of BrdU were performed prior to collection of samples for in vitro study. Wholemount preparations labeled with an antibody towards BrdU clearly show increased proliferation in the ephrin-A5-Fc infused animals. The BrdU-positive cells in the infused animals have a clustered hyperplasia-like appearance. The increased proliferation could be a result of the infused ephrin-A5 interfering with endogenous Eph-Ephrin signaling. Unclustered EphA7-Fc proteins appear to be able to induce the same effect.

If plated on a surface coated with EphA7-Fc, the neurosphere response will depend on whether the EphA7-Fc protein is preclustered or not, indicating a ligand signaling pathway. Poly-0-lysine coated surfaces were coated with EphA7-Fc in preclustered or unclustered conformation. Neurospheres allowed to attach and differentiate exhibited diametrically different behavior depending on whether the EphA7-Fc proteins were preclustered or not. Cells in neurospheres seeded on unclustered EphA7-Fc displayed fast and increased migration and differentiation along with increased size of the attached sphere indicating an increase in proliferation. Cells in neurospheres seeded on clustered EphA7-Fc showed none or minimal migration, differentiation or proliferation.

These spheres remained small and undifferentiated with a rounded morphology. The difference in neurosphere response to clustered vs. unclustered EphA7 indicates a signaling pathway that goes in the reverse direction, that is through the ephrin-A5 and/or ephrin-A2 ligand. Taken together with the examples mentioned above we believe that these results can best be explained with a model where the Ephrin-A ligand upon receptor binding negatively regulates proliferation in the neurogenic region of the ventricular wall.

### Example 2

### Preparation of Samples

IN-SITU HYBRIDIZATION—For EphA7-FL/T1/T2, ephrin-A5 and ephrin-A2 mRNA expression adult mice were perfused with 4% paraformaldehyde, the brains were put into 10% sucrose overnight. After the overnight incubation, the brain was cryosectioned into slices of 12 μm thick. Digoxygenin-labeled riboprobes complementary to the targeted genes were used according to well know in situ hybridization methods such as those described in Henrique et al., (1995).

BRDU-LABELING AND IMMUNOHISTOCHEMISTRY—Adult mice received three intraperitoneal injections of BrdU with two hour intervals and were then sacrificed and perfused with 4% paraformaldehyde. After dissection, the brains were post-fixed for between one and two hours and put into 10% sucrose overnight. The brains were either cryosectioned 12 μm thick or processed for wholemount labeling using common techniques such as those described in Conover et al., (2000). For immunohistochemistry on the cyrosections, antisera against Bromodeoxyuridine (BrdU) (BD Biosciences Pharmingen, San Diego, Calif.) was used at a dilution of 1:200 and visualized with an anti-mouse alexa-488 secondary antibody at a dilution of 1:500. Wholemount BrdU-labeling was performed using common techniques such as those described in (Conover, J. C. et al., 2000. Nat Neurosci 3: 1091-1097).

NEUROSPHERE CULTURES—Neurosphere cultures from adult mice were prepared using techniques described in Johansson et al., 1999. Cell 96: 25-34.

INTRAVENTRICULAR INFUSION—Osmotic pumps filled with either ephrin-A5-Fc (200 μg/ml) or EphA7-Fc (200 μg/ml) fusion proteins were fitted on wild type adult mice as previously described (Conover, J. C. et al., 2000. Nat Neurosci 3: 1091-1097).

The investigation of the role of relevant ligands/receptors in vivo using healthy and/or models for disease/trauma/disorders will be conducted according to the following protocol (intravenous administration), here described for rats, but available also for mice:

NEUROGENESIS—In vivo testing of compounds. The animals used were commercially purchased male rats and mice.

ANIMAL HUSBANDRY—Animals were housed in a regiment of 12 hours light/12 hours darkness and were fed standard pellets with food and water provided ad libitum. Rats were housed in the standard capacity of 5 animals per standard cage;

COMPOUND ADMINISTRATION—Brain infusion was performed by osmotic mini-pumps. Typical duration of administration is one to 14 days with BrdU or ³H-thymidine or other relevant compounds such as marker of proliferation. The animals were studied for 0-4 weeks post infusion. Animal handling and surgery were performed as described as in Pencea V et al., J. Neurosci Sep. 1 (2001), 21(17):6706-17.

REMOVAL OF PUMPS—Pumps were removed after 1 to 14 days under proper anesthesia of the animals.

SAMPLE COLLECTION—Animals were transcardial perfused with NaCl until cessation of vital signs. This was followed by perfusion with a 4% paraformaldehyde solution. Brains were removed and stored in 4% paraformaldehyde overnight and transferred to 30% sucrose solution at 4° C. The bulbus olfactorius (OB) was separated from the rest of the brain. Freezing was performed by submersion in −80° C. (in liquid methylbutane) and long term storage was performed in the −80° C. freezer.

SECTIONING—Sagittal sectioning of ipsilateral OB and coronal sectioning of rest of brain on cryotome.

### Example 3

### Biopolymer Sequences

The DNA and protein sequences referenced in this patent are as listed below. These sequence Genbank accession numbers are also listed.

Mouse EphA7

BC026153 Mus musculus, clone MGC:14056 IMAGE:3991628, mRNA, complete cds

X79082 M.musculus mRNA for kinase 1

X81466 M.musculus mRNA for Ebk receptor tyrosine kinase

X79083 M.musculus mRNA for kinase 1, truncated variant 1

X79084 M.musculus mRNA for kinase 1, truncated variant 2

Human EphA7

L36642 Homo sapiens receptor protein-tyrosine kinase (HEK11) mRNA, complete cds

NM_{—}004440 Homo sapiens EphA7 (EPHA7), mRNA

Mouse ephrin-A2, Efna2

U14941 Mus musculus ELF-1 precursor mRNA, complete cds

U14752 Mus musculus Cek7 ligand mRNA, complete cds

NM_{—}007909 Mus musculus ephrin A2 (Efna2), mRNA

Human ephrin-A2, EFNA2

AJ007292 Homo sapiens mRNA for ephrin-A2

NM_{—}001405 Homo sapiens ephrin-A2 (EFNA2), mRNA

Mouse ephrin-A5, efna5

U90664 Mus musculus ligand AL-1/RAGS mRNA, complete cds

NM_{—}010109 Mus musculus ephrin A5 (Efna5), mRNA

U90665 Mus musculus ligand AL-1s/RAGS mRNA, complete cds

Human ephrin-A5, EFNA5

U26403 Human receptor tyrosine kinase ligand LERK-7 precursor (EPLG7) mRNA, complete cds

NM_{—}001962 Homo sapiens ephrin-A5 (EFNA5), mRNA

### Example 4

### Human Stem Cell (HSC) Cultures

A biopsy from the anterior lateral wall of the lateral ventricle was taken from an adult human patient and enzymatically dissociated in PDD (Papain 2.5 U/ml; Dispase 1 U/ml; Dnase 1250 U/ml) in DMEM containing 4.5 mg/ml glucose and 37° C. for 20 min. The cells were gently triturated and mixed with three volumes of Human Stem Cell Plating Medium (HSCPM) (DMEM/F12; 10% FBS). The cells were pelleted at 250×g for 5 min. The supernatant was subsequently removed and the cells resuspended in HSCPM, plated out on fibronectin coated culture dishes and incubated at 37° C. in 5% CO₂. The following day the expansion of the culture was initiated by change of media to HSC culture media (DMEM/F12; BIT 9500; EGF 20 ng/ml; FGF2 20 ng/ml). The HSC were split using trypsin and EDTA under standard conditions. FBS was subsequently added to inhibit the reaction and the cells collected by centrifugation at 250×g for 5 min. The HSC were replated in HSC culture media.

### Example 5

### In Vivo Testing of Ephrin-A5-FC

Male rats (12 hours light/dark regime; feeding and drinking ad libitum; 5 animals in standard cage) were infused (Alzet minipumps) in the left lateral ventricle with human recombinant ephrin-A5-FC(R&D systems Inc, USA, MN) for 14 days at a daily dose of 2.4 μg/day (8 animals/group). Bromodeoxyuridine (BrdU) was also included in the infusion vehicle (artificial cerebrospinal fluid) to enable measurement of proliferation by quantification of BrdU incorporation in the DNA. Animals were sacrificed at 28 days after start of treatment and brains were dissected and prepared for sectioning and immunohistochemistry (Pencea V et al., J. Neurosci Sep. 1 (2001), 21(17):6706-17).

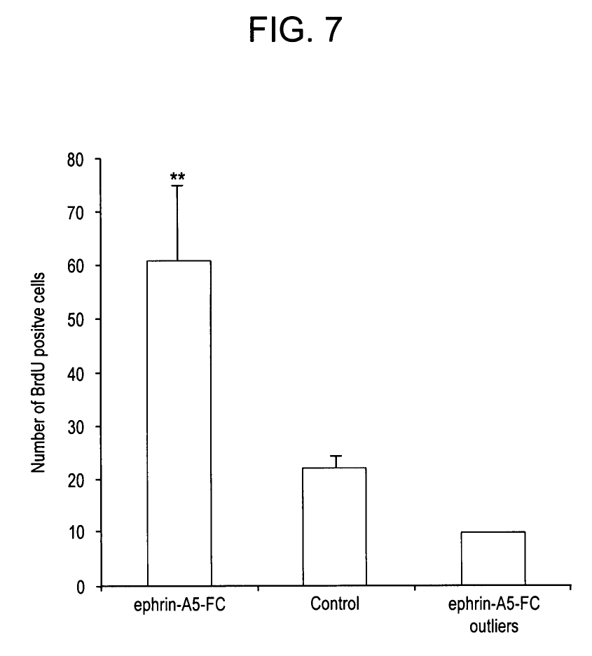

Proliferation was measured by BrdU incorporation and diaminobenzidine (DAB) staining of HRP conjugated secondary antibodies (FIG. 7). Cells were counted in a phase contrast microscope. Neural phenotype is estimated to at least 84% of the newborn cells in olfactory bulb (Petreanu L and Alvarez-Buylla A, J. Neurosci. Jul. 15 (2002), 22(14):6106-13).

### Example 6

### RT-PCR

RT-PCR—Total-RNA was isolated from neurospheres and dissected lateral ventricular wall tissue with the RNeasy™ kit (Qiagen). Reverse transcription was performed with Superscript-II [Invitrogen] and the cDNA was amplified with primers specific for the Ephrin-A & Bs and the EphA & Bs.

The following primer pairs were designed to specifically identify the presence of EphA7 (Gene bank Acc no L36642), ephrin-A5 (Gene bank Acc no U26403), and ephrin-A2 (Gene bank Acc no AJ007292) gene expression in human stein cell cultures. Estimated band sizes for each primer pair are given below:

Band size
(base pairs)

| | | | |
|---|---|---|---|
| EphA7 | 5′-TGGACAGCAC | (SEQ ID NO:1) | 517 |
| | CCGAAGCCAT-3′ | | |
| | 5′-GATGACCAAC | (SEQ ID NO:2) | |
| | CAGTGTGATC | | |
| | CCT-3′ | | |
| EphA7 | 5′-AAAAAGCTAA | (SEQ ID NO:3) | 347 |
| | ACGTGGAGCA | | |
| | GCC-3′ | | |
| | 5′-CCATTGGGTG | (SEQ ID NO:4) | |
| | GAGAGGAAA | | |
| | TCC-3′ | | |
| ephrin-A5 | 5′-GATTCCTTTT | (SEQ ID NO:5) | 375 |
| | TTCTCCTGAA | | |
| | CCC-3′ | | |
| | 5′-TTCCAGTAGA | (SEQ ID NO:6) | |
| | CAGCGTAGC | | |
| | GGT-3′ | | |
| ephrin-A5 | 5′-GATTCCTTTT | (SEQ ID NO:7) | 509 |
| | TCCTCCTGAA | | |
| | CCC-3′ | | |
| | 5′-CCATGTAGAG | (SEQ ID NO:8) | |
| | GACATAGCGC | | |
| | TCA-3′ | | |
| ephrin-A2 | 5′-CGCTGCTGCT | (SEQ ID NO:9) | 363 |
| | CCTGCTGTTA-3′ | | |
| | 5′-GGAACTTCTC | (SEQ ID NO:10) | |
| | CGAGAACTTG | | |
| | AGC-3′ | | |
| ephrin-A2 | 5′-CGCTGCTGCT | (SEQ ID NO:11) | 509 |
| | CCTGCTGTTA-3′ | | |
| | 5′-CTCGTACAGG | (SEQ ID NO:12) | |
| | GTCTCGTTG | | |
| | GTC-3′ | | |

Human stem cells (HSC) were prepared and cultured as stated above. Total RNA isolated using Qiagen's RNeasy Mini Kit according to the manufacturer's instructions and DNase treated using Ambion DNase I and according to protocol. Life Technology's One-Step RT-PCR Kit was used to detect the presence of EphA7, ephrin-A5 and ephrin-A2 mRNA. Briefly, 50 ng of total RNA was used in each reaction, with an annealing temperature of 55° C. To further ensure that genomic contamination of the total RNA did not give rise to false positive results, an identical reaction in which the RT-taq polymerase mix was replaced by taq polymerase alone and was run in parallel with the experimental RT-PCR. The reactions were electrophoresed on a 1.5% agarose gel containing ethidium bromide and the bands visualised under UV light. Bands corresponding to the estimated length of PCR products of the desired genes were cloned into the cloning vector pCR II TOPO (Invitrogen) and sequenced to verify their identity.

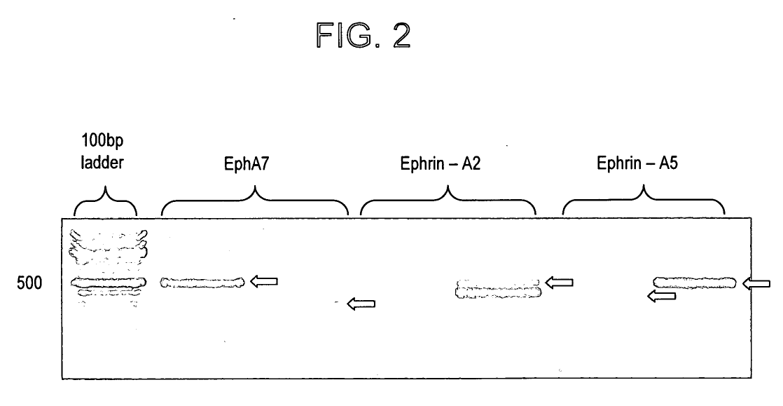

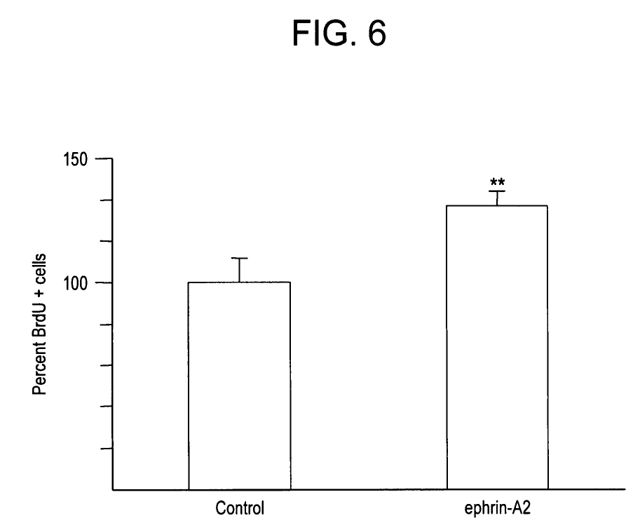

The results of this experiment may be seen in FIG. 2. In FIG. 6, EphA7, ephrin-A2 and ephrin-A5 genes are expressed in cultured Human Neural Stem Cells. RT-PCR was performed on total RNA prepared from cultured HSC using primer pairs specific for the above genes. The bands indicated with an arrow correspond to the bands of the desired size (EphA7 [lane2 517 bp; lane3 347 bp], ephrin-A2 [lane4 no product; lane5 509 bp], ephrin-A5 [lane6 363 bp; lane7 509 bp]), verifying that they represent correct product. A parallel control experiment without using any reverse transcriptase, only taq polymerase, ruled out false positive bands through genomic contamination.

### Example 7

### Immunohistochemistry

Analysis and quantification will be done for proliferative brain regions, migratory streams and areas of clinical relevance (some, but not all, of these areas are exemplified below).

DAB (diamine benzidine) or fluorescence visualization using one or several of the following antibodies: as neuronal markers NeuN, Tuj1, anti-tyrosine hydroxylase, anti-MAP-2 etc.; as glial markers anti-GFAP, anti-S100 etc.; as oligodendrocyte markers anti-GalC, anti-PLP etc. For BrdU visualization: anti-BrdU.

Quantification:

I) DAB-BrdU-Immunohistochemistry

Stereological quantification in ipsilateral brain regions

II) 4-weeks-survival-group: ipsilateral hemisphere

a) Quantification of BrdU+cells via DAB-Immunohistochemistry (stereology)

dorsal hippocampus dentate gyrus

dorsal hippocampus CA1/alveus

olfactory bulb (OB)

subventricular zone (SVZ)

striatum

b) Quantification of double-staining with confocal microscope for every (OB, DG, CA1/alveus, SVZ, wall-to-striatum) structure: checking of BrdU+ for double-staining with the lineage markers. For further experimental details, see Pencea V et al., J. Neurosci Sep. 1 (2001), 21(17):6706-17.

CLUSTERING OF EPHA7 AND COATING—EphA7-Fc fusion protein (R&D systems) were clustered with anti-human IgG (Jackson) as previously described (Davis et al., 1994). Plastic petri dishes were coated with poly-0-ornthinine O/N at 37° C. and then coated with clustered or unclustered EphA7-Fc (10 μg/ml) O/N at 37° C. Neurospheres were then seeded onto the plates and subsequently analyzed with light-microscopy.

In vivo experiments to define the therapeutic potential of ephrin-A2 or -A5 and their receptor EphA7

Ultimately the identification of proliferating and differentiating factors is likely to provide insights into the stimulation of endogenous neurogenesis, in the adults, for the treatment of neurological diseases and disorders. A role for tyrosine kinases (RTKs) in neurogenesis and neuronal differentiation has begun to emerge. In particular, Eph receptor and ephrin ligand signaling has recently been explored to have role in these processes (Miao, H., B. R. Wei, et al. (2001) Nat Cell Biol 3(5): 527-30, Conover, J. C., F. Doetsch, et al. (2000) Nat Neurosci 3(11): 1091-7).

To gain evidence as to whether receptors Ephs or their ligands ephrins (e.g. EphA7 ephrin-A5 or ephrin-A2) can stimulate neurogenesis, mediated through interacting and/or interrupting binding through receptor and/or ligand, in vivo studies is conducted. A number of studies have been carried out testing the potency of growth factors to influence neurogenesis by the method of intraventricular infusion. Infusion of both EGF and basic FGF have been shown to proliferate the ventricle wall cell population, and in the case of EGF, extensive migration of progenitors into the neighboring striatal parenchyma (Craig, C. G., V. Tropepe, et al. (1996) J Neurosci 16(8): 2649-58; Kuhn, H. G., J. Winkler, et al. (1997) J Neurosci 17(15): 5820-9.) Differentiation of the progenitors was predominantly into a glial lineage while reducing the generation of neurons (Kuhn, H. G., J. Winkler, et al. (1997) J Neurosci 17(15): 5820-9.). A recent study found that intraventricular infusion of BDNF in adult rats promotes increases in the number of newly generated neurons in the olfactory bulb and rostral migratory stream, and in parenchymal structures, including the striatum, septum, thalamus and hypothalamus (Pencea, V., K. D. Bingaman, et al. (2001). J Neurosci 21(17): 6706-17.).

A new study (Neuronova, unpublished results) utilizing intraventricular infusion of the receptor EphA7 and the ligand ephrinA2-Fc fusion proteins confirms the increase in proliferation that was observed after the first round of experiments. In addition to the clustered and unclustered EphA7 and ephrin-A5 proteins, ephrinB1 was included as an extra control. The control used in the previous experiment, the anti-Fc antibody, was also included. In the study performed by Conover, J. C., F. Doetsch, et al. (2000), ephrin-B1 elicited no proliferative effect and this is confirmed by the results in our recent round of experiments. If the results of both experiments are taken into account an increase in BrdU-incorporation in the lateral wall was observed.

However, the initial idea that clustered EphA7 would be able to activate ephrin-A2 and thus suppress proliferation can clearly be challenged. This can be due to failure of activation of ephrin-A2 by the clustered EphA7 complex in solution or if it some other signaling properties of the proliferating cells. It appears as if both clustered and un-clustered EphA7 and ephrin-A2 are able to disrupt endogenous signaling and increase proliferation.

Interestingly there appears to be a slight (1.25×) increase of tunnel-positive (TdT-mediated dUTP digoxigenin nick end labeling) cells (apoptosis) in the lateral wall of mice infused with clustered ephrin-A2. This indicates that at least the clustered ephrin-A2 is able to exert other effects than blocking endogenous ephrin signaling. The in vitro results show increased proliferation of neurospheres of material from EphA7 and ephrin-A2 knockout animals. Both the number of sphere-forming cells and the proliferative capacity of those cells are increased. This is in line with the increased number of BrdU⁺ cells in the lateral wall of EphA7 and ephrin-A2 knockouts. In addition to this increase double staining with the mitotic marker (PCNA) and BrdU reveals a shortened cell cycle in these knockouts. The mechanisms underlying these changes in proliferation capacity remain unsolved but the results indicate that disruption of endogenous EphA7/ephrin-A2 signaling is a crucial component. This notion is supported both by data from the knockouts, the Eph/ephrin infusions and the in vitro data.

To determine the effects on neurogenesis, unclustered and/or clustered EphA7, ephrin-A5, ephrin-A2 or alternative binding proteins, derivatives, orthologs, paralogs, mimetics, small molecular weight compounds, antibodies or affibodies will be intraventricularly infused at a range of concentrations into mice and/or rats. The basic experimental set up for infusion of unclustered and/or clustered EphA7, ephrin-A5, ephrin-A2 or alternative binding molecules (see above) into the rodent lateral ventricle and the detection of new neurons and glia is described below.

Further evidence for the importance of ephrins and their receptors can be gained by the use of knockout mice for these proteins (see examples below) is shown. Indeed, targeted deletion of EphA7 in mice indicates an increased proliferation in lateral ventricular wall. Furthermore, more evidence can be gained by using EphA7, ephrin-A5 and Ephrin-A2 knockout mice singularly or in combination, together with animal disease models (see list below), that can improve or worsen the state of the disease model.

In addition to determine the effects of ephrin-A5 or ephrin-A2 that function through the EphA7 receptor family, in healthy animals, it is ultimately the treatment of diseases and disorders through stimulation of neurogenesis that is the goal. The list of diseases that may benefit from increased neurogenesis is extensive, including Parkinson's, Alzheimer's, all forms of depression, schizophrenia, Huntington's, and disorders such as spinal cord injury. To this aim, the above selection of Eph-A7, ephrin-A5 and ephrin-A2 or related Eph receptors and binding compounds (see above) may be applied by intraventricular infusion in rodent and non-human primate disease models as potential treatments. Models for Parkinson's in rodents include MPTP or 6OHDA treatment.

The intraventricular infusion of unclustered and/or clustered Eph-A7, ephrin-A5, ephrin-A2 or alternative binding proteins, derivatives, orthologs, paralogs, mimetics, small molecular weight compounds, antibodies or affibodies, essentially bypasses systemic side effects of the applied compound. Successful results from the above experiments will be carried out to assess this application approach.

Furthermore, the crystal structure of Eph-A7, ephrin-A5, ephrin-A2 singularly or in complex can be used for structure based drug design or structure based in silico screening. Recent publications have revealed the crystal structure of the receptor Eph-B2 in complex with the ligand ephrin-B2 (Himanen J. P., K. R. Rajashankar et al 2001. Nature 414(6866): 933-8; Himanen J P and D. B. Nikolov. 2002. Acta Crystallogr D Biol Crystallogr 58(Pt 3): 533-5). This information will facilitate the development of homology structures of EphA7, ephrin-A5, ephrin-A2, that can be used in the development of derivatives, mimetics, small molecular weight compounds, antibodies or affibodies as well as dissecting the biological functionalities of the ligand-receptor pairs.

In summary, the results of the work presented here suggest that the EphA7 and ephrinA5 could be used for therapeutic applications in the treatment of CNS conditions.

We have shown that the EphA7/ephrinA5-A2 system is expressed in neurogenic areas of the adult mouse brain, and also by neurospheres derived from the lateral ventricular wall. Intraventricular infusion of unclustered (=inactivating) ephrin-A5 increases the number of newborn cells in the lateral ventricular wall of these mice. Infusion of unclustered EphA7 proteins has the same effect. This indicates that interfering with the normal Eph-ephrin signaling (both on the receptor and the ligand side) releases the proliferation block, resulting in increased proliferation. We therefore propose that proteins, peptides, small molecules, antibodies or affibodies that interact with ephrinA2, A5 or EphA7 and block the normal signaling can be used to enhance neurogenesis in the adult brain. Conditions such as neurodegenerative disease, depression, stroke, traumatic injury to the CNS are candidate indications for treatments based on stimulated neurogenesis.

Neural stem cell cultures express EphA7 and ephrinA5/A2. We have shown that the rate of proliferation, migration and differentiation of these neurospheres in vitro is dependent on and can be manipulated through the Eph/ephrin system. Possible applications for these findings may be in the propagation and/or differentiation of neural stem cells for use in transplantation as well as for developing in vitro model systems for pharmacological testing.

### Example 8

### Animal Models

The following animal models of CNS disease/disorders/trauma are to be used to demonstrate recovery. The following examples are not meant to be limiting; additional/modified models will also be used:

Models of epilepsia, such as: Electroshock-induced seizures (Billington A et al., Neuroreport 2000 Nov. 27; 11(17):3817-22), pentylene tetrazol (Gamaniel K et al., Prostaglandins Leukot Essent Fatty Acids 1989 February; 35(2):63-8) or kainic acid (Riban V et al, Neuroscience 2002; 112(1): 101-11) induced seizures

Models of psychosis/schizophrenia, such as: amphetamine-induced stereotypes/locomotion (Borison R L & Diamond B I, Biol Psychiatry 1978 April; 13(2):217-25), MK-801 induced stereotypies (Tiedtke et al., J Neural Transm Gen Sect 1990; 81(3):173-82), MAM (methyl azoxy methanol-induced (Fiore M et al., Neuropharmacology 1999 June; 38(6):857-69; Talamini L M et al., Brain Res 1999 Nov. 13; 847(1):105-20) or reeler model (Ballmaier M et al., Eur J Neurosci 2002 April; 15(17):1197-205)

Models of Parkinson's disease, such as: MPTP (Schmidt & Ferger, J Neural Transm 2001; 108(11):1263-82), 6-OH dopamine (O'Dell & Marshall, Neuroreport 1996 Nov. 4; 7(15-17):2457-61) induced degeneration

Models of Alzheimer's disease, such as: fimbria fomix lesion model (Krugel et al., Int J Dev Neurosci 2001 June; 19(3):263-77), basal forebrain lesion model (Moyse E et al., Brain Res 1993 Apr. 2; 607(1-2):154-60)

Models of stroke, such as: Focal ischemia (Schwartz D A et al., Brain Res Mol Brain Res 2002 May 30; 101(1-2):12-22); global ischemia (2- or 4-vessel occlusion) (Roof R L et al., Stroke 2001 November; 32(11):2648-57; Yagita Y et al., Stroke 2001 August; 32(8):1890-6)

Models of multiple sclerosis, such as: myelin oligodendrocyte glycoprotein—induced experimental autoimmune encephalomyelitis (Slavin A et al., Autoimmunity 1998; 28(2): 109-20)

Models of amyotrophic lateral sclerosis, such as: pmn mouse model (Kennel P et al., J Neurol Sci 2000 Nov. 1; 180(1-2):55-61)

Models of anxiety, such as: elevated plus-maze test (Holmes A et al., Behav Neurosci 2001 October; 115(5):1129-44), marble burying test (Broekkamp et al., Eur J Pharmacol 1986 Jul. 31; 126(3):223-9), open field test (Pelleymounter et al., J Pharmacol Exp Ther 2002 July; 302(1):145-52)

Models of depression, such as: learned helplessness test, forced swim test (Shirayama Y et al., J Neurosci 2002 Apr. 15; 22(8):3251-61), bulbectomy (O'Connor et al., Prog Neuropsychopharmacol Biol Psychiatry 1988; 12(1):41-51)

Models for learning/memory, such as: Morris water maze test (Schenk F & Morris R G, Exp Brain Res 1985; 58(1):11-28)

Models for Huntington's disease, such as: quinolinic acid injection (Marco S et al., J Neurobiol 2002 March; 50(4):323-32), transgenics/knock-ins (reviewed in Menalled L B and Chesselet M F, Trends Pharmacol Sci. 2002 January; 23(1):32-9).

### Example 9

### Production of Mutant Mice

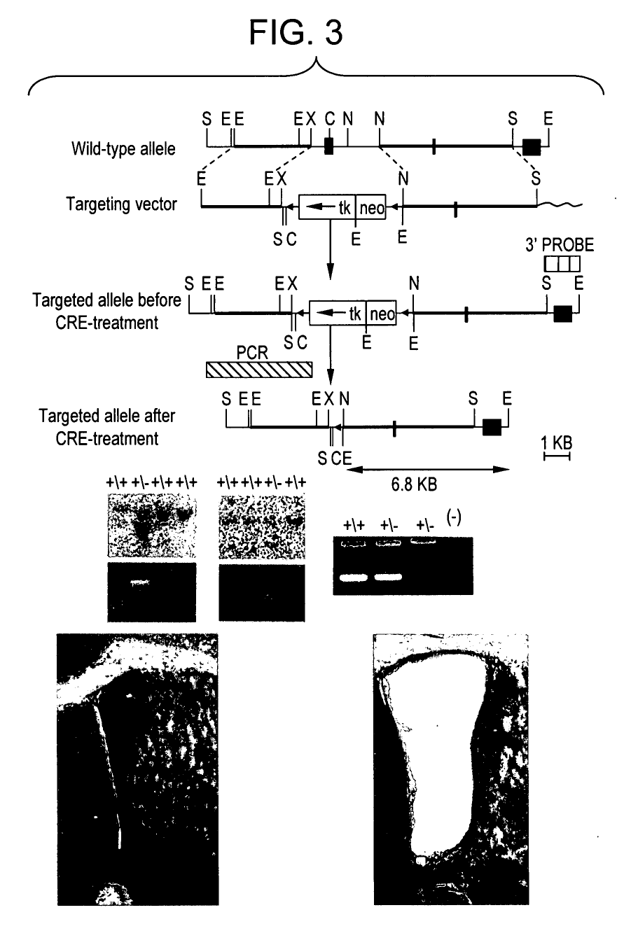

For homologous recombination, 5′EcoRI-XhoI 3-kb sequence and 3′ NotI-SalI 5.3-kb sequence flanking exon I (1-330 bp of the EphA7 cDNA) including part of the upstream sequence (−601 to −1) were subcloned into pBluescript vector (Stratagene, CA). A loxP-flanked selection cassette containing a neomycin-resistance gene and the thymidine kinase gene (tk/neo), both with the phosphoglycerate promoter and polyadenylation signal, was inserted by cloning between these genomic sequences. The R1 embryonic stem cell line was electroporated with the linearized targeting construct and selected with G418 for 10 days. A total of 360 clones were expanded, and homologous recombinants were identified by Southern blot analysis of genomic DNA from single clones digested with EcoRI. See FIG. 3H.

The 5′ end of the targeted allele was checked for integrity using 5′-CTTGACAGCTAAATATCTGGATAAAGAGATC-3′ (SEQ ID NO:13) sense and 5′-CATTACACTTCCAGACCTGGGAC-3′ (SEQ ID NO:14) reverse primer generating a 3.6-kb band in case of correct homologous recombination. From the 12 resulting positive clones, three were transfected with the expression plasmid pIC-Cre coding for Cre recombinase in order to remove the loxP-flanked tk/neo selection cassette. Clones were counter-selected with the thymidine kinase substrate gancyclovir (2 M). Surviving clones were expanded and tested with the genomic probe as described above.

To analyze the removal of the loxP-flanked tk/neo selection cassette, genomic DNA was tested in a PCR reaction using 5′-CTAAGGTCCTATTTTGCCTG-3′ (SEQ ID NO:15) sense primer and the reverse primer described above, leading to the amplification of a 0.5-kb band from the targeted allele. Primers used in RT-PCR for demonstrating the absence of the signal peptide of EphA7 in transgenic animals were 5′-GTCTGCAGTCGGAGACTTGCAG-3′ (SEQ ID NO:16) and 5′-CTTCGCAGCCTGCGCCTC-3′ (SEQ ID NO:17), amplifying a 314-bp band from the 5′-region of the EphA7 mRNA. EphA7 null mice displaying neural tube defects die immediately after birth and were not included in the analysis. EphA7 mutant mice were genotyped by PCR. The strain had a mixed 129/Sv and C57/b16 genetic background and wild type littermates were used as controls in all experiments.

### Example 10

### In Situ Hybridization, Immunohistochemistry and Ephrin/Eph-Fc Labeling

Digoxygenin-labeled riboprobes complementary to ephrin-A2, EphA7 (bases 2601-2925) [Ciossek T. et al., Oncogene. 1995 Nov. 16; 11(10):2085-95) were used for in situ hybridization as described (Schaeren-Wiemers, N., and Gerfin-Moser, A. (1993) A single protocol to detect transcripts of various types and expression levels in neural tissue and cultured cells: in situ hybridization using digoxigenin-labeled cRNA probes. Histochemistry 100, 431-440.). Ephrin-A2 and EphA7-Fc binding was performed after the principle of Cheng and Flanagan [Cheng H. J. and Flanagan J. G., Cell. 7; 79(1):157-68).

Tissue dissociation and culture conditions were essentially as described in Johansson, C. et al. 1999. Cell 96: 25-34). Neural stem cells were passaged by dissociating neurospheres by using trypsin see Johansson C. et al. 1999. Cell 96: 25-34. Differentiation of the neural stem cells was induced by plating on poly-o-ornithine-coated slides.

CELL PROLIFERATION—BrdU (65 mg/kg in 0.9% NaCl, Sigma-Aldrich) was delivered by a single intraperitoneal injection, and was detected with mouseαBrdU in 14 μm cryostat sections.

EPHRIN-A2-FC DELIVERY—Adult male C57 B1/6 mice (B&K Universal) were anesthetized with 2.5% (v/v) of 2,2,2-tribromethanol (Sigma-Aldrich) and 2-methyl-2-butanol (Fluka), 1:1, in distilled water (10 ml·kg⁻¹, i.p.). Ephrin-A2-Fc (200 μg/ml in Y, R&D systems, USA, MN) or vehicle was delivered with an osmotic pump (Alzet 1007D, delivering 0.5 μl/h) connected to a canula stereotaxically inserted 0.5 mm posterior and 0.7 mm lateral to Bregma, 2 mm below the dura mater in the right lateral ventricle.

### Example 11

### Confirming the Results in Mice

Analysis of the expression of all A type ephrins and their EphA receptors in the mouse brain revealed prominent expression of ephrin-A2 and EphA7 mRNA and protein in cells of the lateral ventricle wall (FIG. 1). In addition, low levels of EphA4 mRNA were detected by RT-PCR and in situ hybridization and very low levels of protein were seen in the lateral ventricle wall by immunohistochemistry with an antibody against EphA4. Neural stem cells reside in proximity to the lumen of the ventricular system both during embryogenesis and in the adult brain (McKay R., 1997. Science. 276(5309):66-71; Doetsch, F. et al., 1999. Cell 97: 703-716; Johansson, C. et al., 1999. Cell 96: 25-34; Gage F. H., 2000. Science. 287(5457):1433-8; Rietze R. L. et al., 2001. Nature 412(6848):736-9; Capela A and Temple S. 2002 Neuron. 35(5):865-75). EphA7 is expressed in the ventricular zone already at embryonic day 12.5, but expression of A ephrins in this region cannot be detected until late in embryonic development (Rogers J. H. et al., 1999. Brain Res Mol Brain Res. 74(1-2):225-30; Zhang, J. H. et al., 1996. J. Neurosci. 16, 7182-7192). In the adult mouse brain, ephrin-A2 expression is restricted to the subventricular zone, whereas EphA7 is expressed by cells both in the ependymal layer and in the subventricular zone (FIG. 1a-b & d-e). This expression pattern appear to be evolutionarily conserved, with ephrin-A2 expression in the ventricular zone starting late in embryogenesis in macaque monkeys (Donoghue M. J. and Rakic P., 1999. J. Neurosci. 19(14):5967-79) and expression of ephrin-A2 and EphA7 mRNA in the adult human lateral ventricle wall (data not shown).

The expression of ephrin-A2 and EphA7 in a neural stem cell niche prompted us to generate mice carrying a null mutation in the EphA 7 gene by homologous recombination in embryonic stem cells (FIG. 3i) to elucidate the role of these genes in neurogenesis. EphA7 null mice are born at a slightly lower frequency (24%) than expected from Mendelian inheritance due to prenatal death caused by neural tube defects analogous to that found in subpopulation of ephrin-A5−/− mice (Holmberg J. et al., 2000., Nature 408, 203-206). However, the majority of EphA7 null mice does not display neural tube defects or any other overt phenotype but reaches adulthood and are fertile.

Ephrins, and potentially unknown Eph receptor binding proteins, can be detected with chimeric proteins consisting of the ectodomain of the Eph receptor fused to the Fc part of an immunoglobulin (Eph-Fc) (Cheng, H.-J., and Flanagan, J. G. 1994. Cell 79, 157-168; Gale, N. W. et al., 1996. Neuron 17, 9-19). Detection of EphA7 binding proteins in the lateral ventricle wall with EphA7-Fc revealed a pattern mimicking that of ephrin-A2 expression. Similarly, ephrin binding proteins can be visualized by chimeric ephrin-Fc proteins and ephrin-A2-Fc labeling resulted in a pattern resembling that of EphA7 expression, which was abolished in EphA7−/− mice, arguing that EphA7 is the predominant ephrin-A2 receptor expressed in this part of the brain. However, low levels of EphA4 expression may partially compensate for the loss of EphA7.

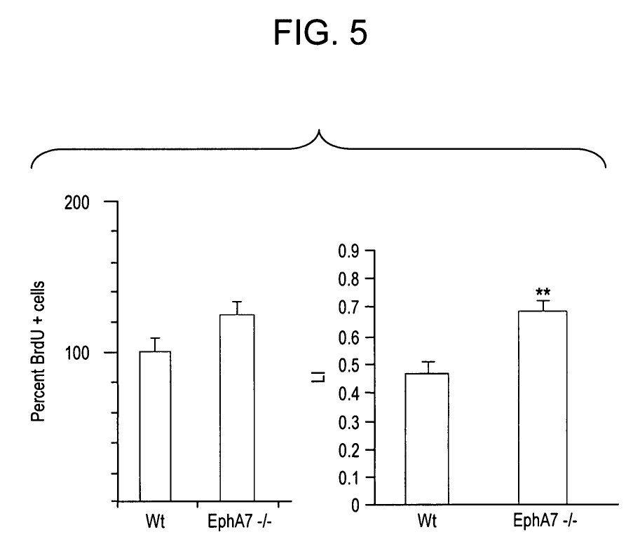

We asked whether ephrin-A2 and EphA7 regulate cell proliferation in the neural stem cell niche. Bromo-deoxyuridine (BrdU) labeling of dividing cells in EphA7−/− mice revealed a 59.1±12.4% (mean ±SEM, P<0.03., n=4) increase (FIG. 5a), respectively, in the number of labeled cells compared to wild type littermates, suggesting that ephrin-A2 and EphA7 are negative regulators of cell proliferation. However, an alternative explanation to the increased number of BrdU labeled cells in the lateral ventricle wall could be that ephrin-A2 and EphA7 inhibit apoptosis, resulting in reduced elimination of newborn cells. We found no significant changes in (TdT-mediated dUTP digoxigenin nick end labeling) TUNNEL-positive cells in the knockout (data not shown).

Ephrins and Eph receptors regulate cell migration in several contexts (Wilkinson, D. G., 2001. Nat Rev Neurosci 2(3): 155-64; Holmberg, J., and Frisen, J., 2002. Trends Neurosci. 25, 239-243; Kullander, K., and Klein, R., 2002. Nat. Rev. Mol. Cell. Biol. 3, 475-486) and the increase in BrdU labeling in the lateral ventricle wall could potentially be a result of newborn cells failing to leave the subventricular zone. Since the increased number of BrdU labeled cells was not accompanied by an increase in cell death, one would expect an expansion of the subventricular zone, which was not seen. Nevertheless, to directly test if the increase in BrdU labeling in the mutant mice was due to increased cell proliferation we assessed the cell cycle length of dividing cells in the lateral ventricle wall. We quantified the proportion of PCNA expressing cells in mutant and wild type mice that were labeled by a total of three pulses of BrdU, with two hour intervals, prior to analysis. Since PCNA is expressed throughout the cell cycle of mitotic cells, whereas BrdU only will be incorporated in nuclei of cells in S phase, a shortening of the cell cycle will result in an increase in the proportion of PCNA expressing cells that are labeled by a pulse of BrdU. We found that 45.6±4.9% (mean ±SEM) of PCNA-immunoreactive cells had incorporated BrdU 8 hours after the injection in wild type mice. The BrdU/PCNA labeling index was 67.7±1.7% (mean ±SEM, P<0.01.) in EphA7−/− mice, demonstrating a 48-68.8% reduction in cell cycle length. The increased cell proliferation in the absence of EphA7 establishes this protein as negative regulators of cell proliferation in the brain.

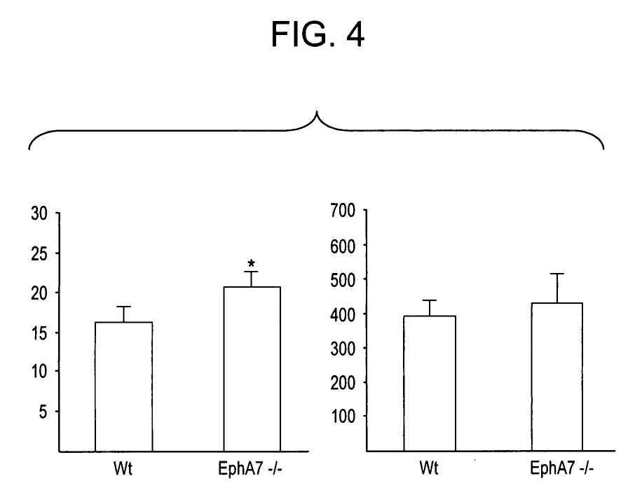

The lateral ventricle wall harbors several distinct cell types including all maturational stages from neural stem cells to neuroblasts (Doetsch, P. et al., 1997. J. Neurosci. 17, 5046-5061). To test if ephrin-A2 and EphA7 regulate the number of neural stem cells, rather than controlling the proliferation exclusively of some other cell population in the ventricular wall, we established primary cell cultures from EphA7−/− mice and wild type littermates and quantified the number of neural stem cell clones (neurospheres). There was no decrease in the number of neurospheres that were able to give rise to all three neural lineages, i.e. neurons, astrocytes and oligodendrocytes from the mutant mice compared to wild type mice, confirming that counted clones indeed derived from neural stem cells. We found a significantly higher number (34-40%) of neurospheres in cultures established from the mutant mice compared to wild type littermates (FIG. 4d), demonstrating an increased number of neural stem cells in the adult brain in the absence of EphA7.

Both ephrin-A2 and EphA7 are expressed in neurospheres, which allowed us to further characterize their role in the regulation of neural stem cell proliferation. We measured cell proliferation in neurospheres, revealing a significant increase in [³H]-thymidine incorporation and cell number in cultures from EphA7−/− mice compared to wild type littermates. However, after a neurosphere is formed from a single neural stem cell, an increasing heterogeneity will ensue as some cells within the clone will gain commitment to certain fates, not making it possible to conclude that the increase in proliferation is in the neural stem cell population rather than in more restricted progenitor cells. To directly assay the number of neural stem cells that were generated in vitro, we quantified the number of cells that could form new neurospheres. We found that the absolute number of secondary neurospheres was higher in EphA7 null compared to wild type cultures. This expansion was greater than anticipated from the increase in cell number (FIG. 4e), suggesting that ephrin-A2 and EphA7 do not only repress neural stem cell proliferation, but also promote the generation of differentiated progeny, at least in vitro. β-catenin, tcf, Pten and Emx2 are examples of modulators of neural stem cell proliferation which may act as intracellular effectors (Chenn, A., and Walsh, C. A., 2002. Science 297, 365-369; Megason, S. G., and McMahon, A. P., 2002. Development 129, 2087-2098; Groszer, M et al., 2001. Science 294, 2186-2189; Galli, R. et al., 2002. Development 129, 1633-1644) although none of these proteins have been reported to interact with or be regulated by Eph receptors.

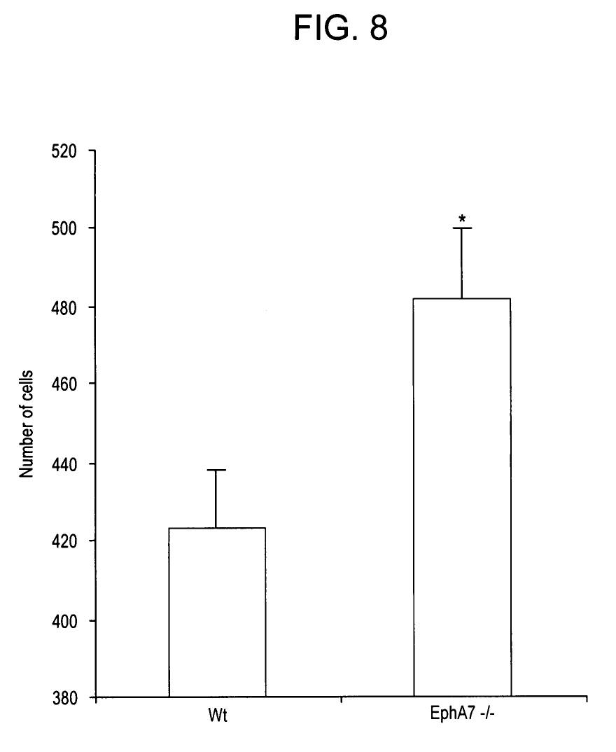

We next analyzed the consequence of increased stem cell proliferation on the number of cells in the brain. The size of the lateral ventricles is drastically reduced in the EphA7−/− mice, leaving only a minimal lumen (FIG. 3, K-M). In spite of the increased cell proliferation in the lateral ventricle wall this does not appear to be a result of a thickening of the ependymal layer or the subventricular zone compressing the ventricle. Detailed histological analysis did not reveal an obvious expansion of any individual brain region, but there rather appears to be a uniform increase in the volume of brain regions resulting in the reduced volume of the lateral ventricle (FIG. 3, K-M). We quantified the number of cells in the brain cortex of wild type, and EphA7 mutant mice (FIG. 8). The 14 μm Cryosections were stained with DAPI to visualize cell nuclei. The nuclei in one 20× microscopic field of a defined area of the cortex were counted. We found that EphA7−/− mice have significantly more cells (mean ±SEM, P<0.05) in their brain cortex compared to wild type littermates. An increase in brain volume during development, for example due to hydrocephalus or null mutations in genes regulating apoptosis or cell intrinsic determinants of proliferation, results in an enlargement of the brain and altered shape of the cranium. If the increase in intracranial volume instead starts late in development, the head shape will not be altered, but an increase in brain volume can only expand into the ventricular system. Ephrin-A2 expression commences late during embryogenesis, and EphA7 null mice do not have an abnormal head shape, although the reduction of the lumen of the lateral ventricle is manifest already at postnatal day 3. Neural stem cell proliferation and neurogenesis drops sharply perinataly, and we conclude that negative regulation by ephrin-A2 and EphA7 contribute to this development.

Cell transplantation is a well-established therapy for several hematopoietic disorders and is a promising approach for the treatment of type I diabetes and Parkinson's disease (Bjorklund, A., and Lindvall, O., 2000. Nature Neuroscience 3, 537-544; Shapiro, A. M. et al., 2000. N. Engl. J. Med. 343, 230-238). Stem cells represent an attractive source for transplantable cells, not least for neuronal replacement (Gage, F. H., 1998. Nature 392 suppl., 18-24; Kim, J. H. et al, 2002. Nature 418, 50-56). An alternative to neuronal replacement by cell transplantation is to stimulate neurogenesis from endogenous stem cells. Several studies have shown that infusion of mitogens can increase cell proliferation in the lateral ventricle wall of the adult brain and in some situations even result in an increase in neurogenesis (Craig, C. G. et al., 1996. J. Neurosci. 16, 2649-2658; Kuhn, H. G. et al., 1997. J. Neurosci. 17, 5820-5829; Nakatomi, H. et al., 2002. Cell 110, 429-441). The identification of ephrin-A2 and EphA7 as negative regulators of neural stem cell proliferation raised the question whether it may be possible to stimulate neurogenesis in the adult brain by blocking the binding of ephrin-A2 to EphA7. Ephrins need to be clustered in the cell membrane to activate Eph receptors, which can be mimicked by clustering recombinant soluble ephrins with antibodies (Davis, S. et al., 1994. Science 266, 816-819). Unclustered soluble ephrins function as antagonists of Eph signaling (Davis, S. et al., 1994. Science 266, 816-819). We delivered unclustered ephrin-A2-Fc directly into the lateral ventricle of adult wild type mice over a three day period via osmotic pumps to test whether we could block the repression of neural stem cell proliferation mediated by the interaction of ephrin-A2 with EphA7. This resulted in a 33.5±7.2% (mean ±SEM, p<0.01, n=7) increase in cell proliferation in the lateral ventricle wall compared to vehicle infused animals (FIG. 6e), approaching the level seen in EphA7 null mice. The increase in cell number in the adult brain achieved by blocking the binding of ephrin-A2 to EphA7 with ephrin-A2-Fc and disrupting the suppression on proliferation establishes inhibition of a negative regulator as a potential therapeutic strategy to expand a stem cell derived population in vivo.

The identification of an extracellular pathway that negatively regulates stem cell proliferation demonstrates a novel control mechanism in a stem cell niche. Ephrins and Eph receptors have recently been identified in screens for genes expression is common to several stem cell populations (Ivanova, N. B. et al., 2002. Science 298, 601-604; Ramalho-Santos, M. et al., 2002. Science 298, 597-600). Interestingly, increased Eph receptor signaling in hematopoietic stem cells by over expression of EphB4, a receptor which is normally expressed in these cells, reduced the number of stem cells in an in vitro assay (Wang, Z. et al., 2002. Blood 99, 2740-2747). Repression of stem cell proliferation by ephrins and Eph receptors may be a general mechanism to control cell number in organs.

1. A method of alleviating a symptom of a disease or disorder of the nervous system comprising administering a modulator to modulate an activity of a neural stem cell or a neural progenitor cell in vivo to a patient suffering from the disease or disorder of the nervous system, wherein the modulator disrupts an interaction between EphA7 and ephrin-A5 or an interaction between EphA7 and ephrin-A2.

2. The method of claim 1 wherein the modulator is administered in an amount of 0.1 ng/kg/day to 10 mg/kg/day.

3. The method of claim 1 wherein the modulator is administered in an amount of 1 ng/kg/day to 10 mg/kg/day.

4. The method of claim 1 wherein the modulator is administered in an amount of 1 ng/kg/day to 5 mg/kg/day.

5. The method of claim 1 wherein the modulator is administered in an amount of 0.1 μg/kg/day to 5 mg/kg/day.

6. The method of claim 1 wherein the modulator is administered to achieve a targeted tissue concentration of 0.1 nM to 50 nM.

7. The method of claim 6 wherein the targeted tissue is selected from the group consisting of tissue adjacent to the lateral ventricular wall, hippocampus, alveus, striatum, substantia nigra, retina, nucleus basalis of Meynert, spinal cord and cortex.

8. The method of claim 6 wherein the targeted tissue is a region of the brain damaged by a disorder, stroke, or ischemia.

9. The method of claim 1 wherein the neural stem cell or neural progenitor cell is a cell that can be isolated from adult bone marrow, spinal cord, epithelial skin, epithelial intestinal, pancreas, hemapoetic system, blood, umbilical cord and muscle.

10. The method of claim 9, wherein said neural stem cell or neural progenitor cell is derived from a pluripotent stem cell contacted to said modulator.

11. The method of claim 1 wherein the modulator is administered by injection.

12. The method of claim 1 wherein the modulator is selected from the group consisting of an EphA7 protein, ephrin-A2, ephrin-A5, a soluble fragment thereof, and an extra-cellular fragment thereof.

13. (canceled)

14. The method of claim 11 wherein the injection is administered orally, subcutaneously, intraperitoneally, intramuscularly, intracerebroventricularly, intraparenchymally, intrathecally or intracranially.

15. The method of claim 1 wherein the modulator is administered to the buccal, nasal or rectal mucosa.

16. The method of claim 1 wherein the modulator is administered via peptide fusion to enhance uptake or via micelle delivery system.

17. The method of claim 1 wherein the disease or disorder of the nervous system is selected from the group consisting of neurodegenerative disorders, neural stem cell disorders, neural progenitor disorders, ischemic disorders, neurological traumas, affective disorders, neuropsychiatric disorders and learning, memory disorders, Parkinson's disease and Parkinsonian disorders, Huntington's disease, Alzheimer's disease, amyotrophic lateral sclerosis, spinal ischemia, ischemic stroke, spinal cord injury, cancer-related brain/spinal cord injury, schizophrenia, psychoses, depression, bipolar depression/disorder, anxiety syndromes/disorders, phobias, stress and related syndromes, cognitive function disorders, aggression, drug and alcohol abuse, obsessive compulsive behaviour syndromes, seasonal mood disorder, borderline personality disorder, cerebral palsy, multi-infarct dementia, Lewy body dementia, age related/geriatric dementia, epilepsy and injury related to epilepsy, spinal cord injury, brain injury, trauma related brain/spinal cord injury, anti-cancer treatment related brain/spinal cord tissue injury, infection and inflammation related brain/spinal cord injury, environmental toxin related brain/spinal cord injury, multiple sclerosis, autism, attention deficit disorders, narcolepsy, retinal degenerative disorders, injury or trauma to the retina and sleet disorders.

18-19. (canceled)

20. The method of claim 1 wherein the neural stem cell or neural progenitor cell activity is proliferation, differentiation, migration or survival.

21. The method of claim 1 wherein the neural stem cell or neural progenitor cell is derived from tissue enclosed by dura mater, peripheral nerves or ganglia.

22. A method of modulating an ephrin receptor or an ephrin ligand on the surface of a neural stem cell or neural progenitor cell comprising the step of exposing the cell expressing the receptor, or ligand to exogenous reagent, antibody, or affibody, wherein the exposure induces the neural stem cell or neural progenitor cell to proliferate, differentiate, migrate or survival.

23-34. (canceled)

35. A method for reducing a symptom of a disease or disorder of the central nervous system in a mammal in need of such treatment comprising administering an ephrin receptor modulator to the mammal, wherein the modulator disrupts an interaction between EphA7 and ephrin-A5 or an interaction between EphA7 and ephrin-A2.

36-86. (canceled)
